(19) United States
(12) Patent Application Publication
(10) Pub. No.: US 2023/0197192 A1
(43) Pub. Date: Jun. 22, 2023

(54) SELECTING NEOANTIGENS FOR PERSONALIZED CANCER VACCINE

(71) Applicant: AMAZON TECHNOLOGIES, INC., Reno, NV (US)

(72) Inventors: David HECKERMAN, Bellevue, WA (US); Frank Wilhelm SCHMITZ, Seattle, WA (US); Michael VOGELSONG, Seattle, WA (US)

(21) Appl. No.: 17/616,145

(22) PCT Filed: Nov. 5, 2021
(86) PCT No.: PCT/US2021/058162

§ 371 Date: Dec. 2, 2021

Related U.S. Application Data
(60) Provisional application No. 63/110,711, filed on Nov. 6, 2020.

(51) Int. Cl. G16B 20/50; C12Q 1/6886

(52) U.S. Cl. CPC G16B 20/50; C12Q 1/6886

(57) ABSTRACT

Disclosed herein are methods for selecting one or more tumor-specific neoantigens from a tumor of a subject for a personalized immunogenic composition. Also disclosed herein are methods for treating cancer in a subject in need thereof by administering an immunogenic composition comprising tumor-specific neoantigens selected using the methods disclosed herein.

The present application claims the benefit of U.S. Provisional Application No. 63/110,711 filed on Nov. 6, 2020, the entire contents of which are incorporated herein by reference.

### REFERENCE TO A SEQUENCE LISTING

This application contains a Sequence Listing in computer readable form. The computer readable form is incorporated herein by reference. Said ASCII copy, created on Oct. 28, 2021, is named 146401_091524_SL.txt and is 75,598 bytes in size.

### 1. BACKGROUND

Cancer is a leading cause of death worldwide accounting for 1 in 4 of all deaths. Siegel et al., CA: A Cancer Journal for Clinicians, 68:7-30 (2018). There were 18.1 million new cancer cases and 9.6 million cancer-related deaths in 2018. Bray et al., CA: A Cancer Journal for Clinicians, 68(6):394-424. There are a number of existing standard of care cancer therapies, including ablation techniques (e.g., surgical procedures and radiation) and chemical techniques (e.g., chemotherapeutic agents). Unfortunately, such therapies are frequently associated with serious risk, toxic side effects, and extremely high costs, as well as uncertain efficacy.

Cancer immunotherapy (e.g., cancer vaccine) has emerged as a promising cancer treatment modality. The goal of cancer immunotherapy is to harness the immune system for selective destruction of cancer while leaving normal tissues unharmed. Traditional cancer vaccines typically target tumor-associated antigens. Tumor-associated antigens are typically present in normal tissues, but overexpressed in cancer. However, because these antigens are often present in normal tissues immune tolerance can prevent immune activation. Several clinical trials targeting tumor-associated antigens have failed to demonstrate a durable beneficial effect compared to standard of care treatment. Li et al., Ann Oncol., 28 (Suppl 12): xii11-xii17 (2017).

Neoantigens represent an attractive target for cancer immunotherapies. Neoantigens are non-autologous proteins with individual specificity. Neoantigens are derived from random somatic mutations in the tumor cell genome and are not expressed on the surface of normal cells. Id. Because neoantigens are expressed exclusively on tumor cells, and thus do not induce central immune tolerance, cancer vaccines targeting cancer neoantigens have potential advantages, including decreased central immune tolerance and improved safety profile. Id.

The mutational landscape of cancer is complex and tumor mutations are generally unique to each individual subject. Most somatic mutations detected by sequencing do not result in effective neoantigens. Only a small percentage of mutations in the tumor DNA, or a tumor cell, are transcribed, translated, and processed into a tumor-specific neoantigen with sufficient accuracy to design a vaccine that is likely to be effective. Further, not all neoantigens are immunogenic. In fact, the proportion of T cells spontaneously recognizing endogenous neoantigens is about 1% to 2%. See, Karpanen et al., Front Immunol., 8:1718 (2017). Moreover, the cost and time associated with the manufacture of neoantigen vaccines is significant.

Thus, it remains a challenge to efficiently and accurately predict, prioritize, and select neoantigen candidates for immunogenic compositions. Accordingly, there is a significant unmet need for an integrated method to characterize tumor genomic material to identify neoantigens, identify which neoantigens are targeted by the immune system, and select which neoantigens are likely to be suitable for effective immunogenic compositions.

### 2. SUMMARY

This disclosure relates to a novel method for selecting one or more tumor-specific neoantigens from a tumor of a subject for a subject-specific immunogenic composition. The disclosure also relates to methods of treating cancer in a subject in need thereof by administering an immunogenic composition comprising tumor-specific neoantigens selected using the novel approach for selecting tumor-specific neoantigens and formulating an immunogenic composition comprising the selected tumor-specific neoantigens. The approach beings with obtaining sequence data from the tumor. The sequence data is used to obtain data representing a polypeptide sequence of one or more tumor-specific neoantigens. The sequence data may be nucleotide sequence data, polypeptide sequence data, exome sequence data, transcriptome sequence data, or whole genome nucleotide sequence data. The sequence data may be whole exome sequence data, RNA sequence data, whole genome sequence data or combinations thereof. The sequence data may be a combination of whole exome sequence data, RNA sequence data, and whole genome sequence data.

The polypeptide sequence(s) and MHC molecule(s) of the subject are then inputted into a machine-learning platform. The machine-learning platform is used to identify whether tumor-specific neoantigens are immunogenic (e.g., that the one or more tumor-specific neoantigen will elicit an immune response in the subject). Based on these predictions, the machine-learning platform generates a numerical probability score that one or more tumor-specific neoantigens will elicit an immune response in the subject.

The MCH molecule(s) of the subject may be MHC class I molecule and/or an MHC class II molecule. The polypeptide sequence encoding one or more tumor-specific neoantigens may be from short polypeptides. Short polypeptides are typically presented on MCH class I molecules. Alternatively, the polypeptide sequence encoding one or more tumor-specific neoantigens can be from long polypeptides.

The immune response in the subject can include presentation of one or more tumor-specific neoantigens to the tumor cell surface, presentation of one or more tumor-specific neoantigens by one or more MHC molecules on the tumor cell, or that one or more tumor-specific neoantigens is capable of presentation to T cells by antigen presenting cells.

The immune response in the subject can be a CD4+ mediated response or a CD8+ mediated response. Typically, the immune response is either a CD4+ mediated response or a CD8+ mediated response.

A tumor-specific neoantigen with a higher numerical probability score relative to a lower numerical probability score indicates that the tumor-specific neoantigen will elicit a greater immune response in the subject.

RNA expression, preferably mRNA expression, of the one or more tumor-specific neoantigens in a tumor is also quantified to further identify one or more tumor-specific neoantigens that are sufficiently expressed to elicit an immune response in the subject. Then, tumor clones can be optionally characterized to ensure that the tumor-specific neoantigens represent sufficient fraction (e.g., genetic diversity) across the tumor. In embodiments, a suitable tumor-specific neoantigen may represent about 1% of the tumor. In other instances, a suitable tumor-specific neoantigen may represent about 5% of the tumor.

These parameters are used to calculate a tumor-specific neoantigen score for the one or more tumor-specific neoantigen scores. The tumor-specific neoantigen score is used to select tumor-specific neoantigens suitable for formulation of a subject-specific immunogenic composition. A higher tumor-specific neoantigen score relative to a lower tumor-specific neoantigen score indicates that the neoantigen has stronger immunogenicity, and thus more likely to induce a strong immune response and elicit stable therapeutic effects (i.e., more likely to be suitable for an immunogenic composition). In embodiments, at least about 10 tumor-specific neoantigens are selected to formulate the subject-specific immunogenic composition. In embodiments, at least about 20 tumor-specific neoantigens are selected to formulate the subject-specific immunogenic composition.

The methods disclosed herein can further comprise measuring the ability of the one or more tumor-specific neoantigens to induce an autoimmune response to normal tissue. A tumor-specific neoantigen that induces an autoimmune response in normal tissue will have a lower tumor-specific neoantigen score relative to a tumor-specific neoantigen that does not induce an autoimmune response. A tumor-specific neoantigen that induces an autoimmune response will not be selected for the immunogenic composition.

The formulated immunogenic composition may include at least about 10 tumor-specific neoantigens or at least about 20 tumor-specific neoantigens. The tumor-specific neoantigens can be encoded by short polypeptides or by long polypeptides. The immunogenic composition may comprise a nucleotide sequence, a polypeptide sequence, RNA, DNA, a cell, a plasmid, a vector, a dendritic cell, or a synthetic long peptide. The immunogenic composition can further comprise an adjuvant.

This disclosure also relates to methods of treating cancer in a subject in need thereof comprising administering a personalized immunogenic composition comprising one or more tumor specific neoantigens selected using the methods described herein. The methods disclosed herein can be suited for treating any number of cancers. The tumor can be from melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Preferably, the cancer is melanoma, breast cancer, lung cancer, and bladder cancer.

### 3. BRIEF DESCRIPTION OF THE DRAWINGS

FIG. 1 is a schematic depicting the approach for selecting one or more tumor-specific neoantigens.

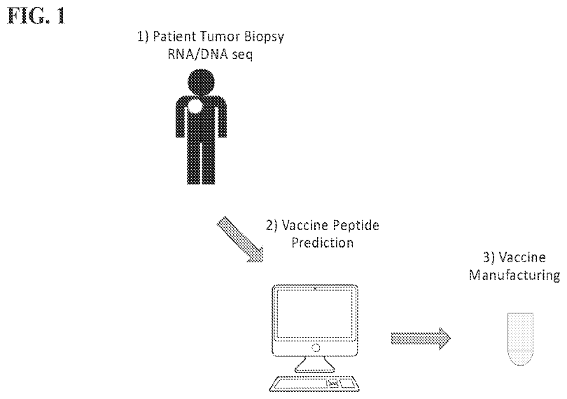

FIG. 2 is schematic flow diagram depicting the bioinformatics analysis of next generation sequencing data (input and output).

FIG. 3 is a flow diagram of the module for clonality deconvolutioion

### 4. DETAILED DESCRIPTION

This disclosure relates to a novel approach for selecting tumor-specific neoantigens with high-accuracy for potent personalized cancer immunogenic compositions (e.g., subject-specific immunogenic compositions). The disclosure also relates to methods of treating cancer in a subject in need thereof by administering an immunogenic composition comprising tumor-specific neoantigens selected using the novel approach for selecting tumor-specific neoantigens and formulating an immunogenic composition comprising the selected tumor-specific neoantigens. The inventors have developed an approach that: 1) sequences the DNA and/or RNA encoding for the polypeptide sequence of one or more neoantigens; 2) determines whether the tumor-specific neoantigen is immunogenic (e.g., whether a neoantigen can elicit an immune response in the subject); 3) determines whether the tumor expresses an amount of neoantigen sufficient to elicit an immune response; and 4) optionally determines whether the neoantigen represents a sufficient fraction of the tumor. Currently available methods rely on MHC binding affinity predictions to rank and select for neoantigens or the probability a neoantigen will be presented by an MHC molecule. These methods do not predict immunogenicity. Moreover, current methods do not have the capability to evaluate all of these factors with high-accuracy.

The approach begins with sequencing the polypeptide sequence of tumor-specific neoantigens obtained from a tumor biopsy. A prediction machine-learning platform is then used to identify which neoantigens are recognized by MHC molecules of the subject. The platform can determine whether the tumor-specific neoantigens are immunogenic (e.g., that the tumor-specific neoantigen will elicit an immune response in the subject). Based on these predictions, the machine-learning platform generates a numerical probability score that the tumor-specific neoantigens will elicit an immune response. RNA expression, preferably, mRNA expression, of tumor-specific neoantigens is also quantified to focus on the tumor-specific neoantigens that are abundantly expressed, such that they will likely elicit an immune response. Then tumor clones are optionally characterized to ensure that the tumor-specific neoantigens represent sufficient genetic diversity across the tumor. These parameters are used to create a tumor-specific neoantigen score for the tumor-specific neoantigens. The tumor-specific neoantigen score is used to select tumor-specific neoantigens suitable for formulation of a personalized vaccine. A higher tumor-specific neoantigen score relative to a lower tumor-specific neoantigen score indicates that the neoantigen has stronger immunogenicity, and thus more likely to induce a strong immune response and elicit stable therapeutic effects.

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “cancer” refers to the physiological condition in subjects in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer, Hormone receptor positive breast cancer), osteosarcoma, melanoma, colon cancer, colorectal cancer, endometrial (e.g., serous) or uterine cancer, salivary gland carcinoma, kidney cancer, liver cancer, prostate cancer, vulvar cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu. Hormone receptor positive breast cancer refers to breast cancer that is positive for at least one of the following: ER or PR, and negative for Her2/neu (HER2).

The term “neoantigen” as used herein refers to an antigen that has at least one alteration that makes it distinct from the corresponding parent antigen, e.g., via mutation in a tumor cell or post-translational modification specific to a tumor cell. A mutation can include a frameshift, indel, missense or nonsense substitution, splice site alteration, genomic rearrangement or gene fusion, or any genomic expression alteration giving rise to a neoantigen. A mutation can include a splice mutation. Post-translational modifications specific to a tumor cell can include aberrant phosphorylation. Post-translational modifications specific to a tumor cell can also include a proteasome-generated spliced antigen. See, Lipe et al., Science, 354(6310):354:358 (2016). In general, point mutations account for about 95% mutations in tumors and indels and frame-shift mutations account for the rest. See, Snyder et al., N Engl J Med., 371:2189-2199 (2014).

As used herein the term “tumor-specific neoantigen” is a neoantigen present in a subject's tumor cell or tissue, but not in the subject's normal cell or tissue.

The term “next generation sequencing” or “NGS” as used herein refers to sequencing technologies having increased throughput as compared to traditional approaches (e.g., Sanger sequencing), with the ability to generate hundreds of thousands of sequence reads at a time.

The term “neural network” as used herein refers to a machine-learning model for classification or regression consisting of multiple layers of linear transformations followed by element-wise nonlinearities typically trained via stochastic gradient descent and back-propagation.

The term “subject” as used herein refers to any animal, such as any mammal, including but not limited to, humans, non-human primates, rodents, and the like. In some embodiments, the mammal is a mouse. In some embodiments, the mammal is a human.

The term “tumor cell” as used herein refers to any cell that is a cancer cell or is derived from a cancer cell. The term “tumor cell” can also refer to a cell that exhibits cancer-like properties, e.g., uncontrollable reproduction, resistance to anti-growth signals, ability to metastasize, and loss of ability to undergo programed cell death.

Additional description of the methods and guidance for the practice of the methods are provided herein.

I. Methods for Selecting Tumor-Specific Neoantigens

Disclosed herein are methods for selecting tumor-specific neoantigens from a tumor of a subject that are suitable for subject-specific immunogenic compositions. Suitable tumor-specific neoantigens are tumor-specific neoantigens that are likely presented on the cell surface of the tumor, are likely to be immunogenic, are predicted to be expressed in sufficient amounts to elicit an immune response in the subject, and optionally represent sufficient diversity across the tumor.

The first step in selecting one or more tumor-specific neoantigens from a tumor of a subject comprises obtaining sequence data from the tumor. The sequence data is used to obtain data representing a polypeptide sequence of one or more tumor-specific neoantigens. Generally, sequence data representing a polypeptide sequence of one or more tumor-specific neoantigens is determined by subjecting a tumor sample to sequence analysis.

The sequence data can be exome sequence data, transcriptome sequence data, whole genome nucleotide sequence data, nucleotide sequence data, or polypeptide sequence data. The sequence data may be whole exome sequence data, RNA sequence data, whole genome sequence data or combinations thereof. The sequence data may be a combination of whole exome sequence data, RNA sequence data, and whole genome sequence data.

Various methods of obtaining sequence data may be used in the methods described herein. Sequencing methods are well known in the art and include, but are not limited to, PCR-based methods, including real-time PC, whole exome sequencing, deep sequencing, high-throughput sequencing, or combinations thereof. In some embodiments, the foregoing techniques and procedures are performed according to the methods described in e.g., Sambrook et al., Molecular Cloning: A Laboratory Manual 4th ed. (2012) Cold Spring Harbor Laboratory Press, Cold Spring Harbor, N.Y. See also, Austell et al., Current Protocols in Molecular Biology, ed., Greene Publishing and Wiley-Interscience New York (1992) (with periodic updates).

Sequencing methods may also include, but are not limited to, high-throughput sequencing, single-cell RNA sequence, RNA sequencing, pyrosequencing, sequencing-by synthesis, single-molecule sequencing, nanopore sequencing, semiconductor sequencing, sequencing-by-synthesis, sequencing-by-ligation, sequencing-by-hybridization, RNA-Sew (Illumina), Digital Gene Expression (Helicos), next generation sequencing, Single Molecule Sequencing by Synthesis (SMSS) (Helicos), massively-parallel sequencing, Clonal Single Molecule Array (Solexa), shotgun sequencing, Maxam-Hilbery or Sanger sequencing, whole genome sequencing, whole exome sequencing, primer walking, sequencing using PacBio, SOLid, Ion Torrent, or Nanopore platforms and any other sequencing methods known in the art. The sequencing method employed herein to obtain sequence data is preferably high-throughput sequencing. High-throughput sequencing technologies are capable of sequencing multiple nucleic acid molecules in parallel, enabling millions of nucleic acid molecules to be sequenced at a time. See, Churko et al., Circ. Res. 112(12):1613-1623 (2013).

In some instances, whole exome sequences, RNA sequencing, whole genome sequencing or combinations thereof can be performed. In some instances, a combination of whole exome sequences, RNA sequencing, whole genome sequencing can be performed.

In some cases, high-throughput sequencing can be next generation sequencing. There are a number of different next generation platforms using different sequencing technologies (e.g., using the HiSeq or MiSeq instruments available from Illumina (San Diego, Calif.)). Any of these platforms can be employed for sequencing the genetic material disclosed herein. Next generation sequencing is based on sequencing a large number of independent reads, each representing anywhere between 10 to 1000 bases of nucleic acid. Sequencing by synthesis is a common technique used in next generation sequencing. In general, sequencing involves hybridizing a primer to a template to form a template/primer duplex, contacting the duplex with a polymerase in the presence of a detectably-labeled nucleotide under conditions that permit the polymerase to add nucleotides to the primer in a template-dependent manner. Signal from the detectable label is then used to identify the incorporated base and the steps are sequentially repeated in order to determine the linear order of nucleotides in the template. Exemplary detectable labels include radiolabels, florescent labels, enzymatic labels, etc. Numerous techniques are known for detecting sequences, such as the Illumina NextSeq platform by cycle end sequencing.

Once sequence data representing the polypeptide sequence of one or more tumor specific neoantigens is obtained, the sequence data, along with the MHC molecule of the subject, is inputted into a machine-learning platform. The machine-learning platform generates a numerical probability score that forecasts whether the one or more tumor-specific neoantigens are immunogenic (e.g. will elicit an immune response in the subject).

MHC molecules transport and present peptides on the cell surface. The MHC molecules are classified as MHC molecules of class I and of class II. MHC class I are present on the surface of almost all cells of the body, including most tumor cells. The proteins of MHC class I are loaded with antigens that usually originate from endogenous proteins or from pathogens present inside cells, and are then presented to cytotoxic T-lymphocytes (i.e., CD8+). The MHC class I molecules can comprise HLA-A, HLA-B, or HLA-C. The MHC molecules of class II are only present on dendritic cells, B lymphocytes, macrophages and other antigen-presenting cells. They present mainly peptides, which are processed from external antigen sources, i.e. outside of the cells, to T-helper (Th) cells (i.e., CD4+). The MHC class II molecules can comprise HLA-DPA1, HLA-DPB1, HLA-DQA1, HLA-DQB1, HLA-DRA, and HLA-DRB1. In some occasions, MHC class II molecules can also be expressed on cancer cells.

MHC class I molecules and/or MHC class II molecules can be inputted into the machine-learning platform. Typically, either MHC class I molecules or MHC class II molecules are inputted into the machine-learning platform. In some embodiments, MHC class I molecules are inputted into the machine-learning platform. In other embodiments, MHC class II molecules are inputted into the machine-learning platform.

MHC class I molecules bind to short peptides. MHC class I molecules can accommodate peptides generally about 8 amino acids to about 10 amino acids in length. In embodiments, the sequence data encoding one or more tumor-specific neoantigens are short peptides about 8 amino acids to about 10 amino acids in length. MHC class II molecules bind to peptides that are longer in length. MHC class II can accommodate peptides which are generally about 13 amino acids in length to about 25 amino acids in length. In embodiments, the sequence data encoding one or more tumor-specific neoantigens are long peptides about 13 to 25 amino acids in length.

The sequence data encoding one or more tumor-specific neoantigens can be about 5 amino acids in length, about 6 amino acids in length, about 7 amino acids in length, about 8 amino acids in length, about 9 amino acids in length, about 10 amino acids in length, about 11 amino acids in length, about 12 amino acids in length, about 13 amino acids in length, about 14 amino acids in length, about 15 amino acids in length, about 16 amino acids in length, about 17 amino acids in length, about 18 amino acids in length, about 19 amino acids in length, about 20 amino acids in length, about 21 amino acids in length, about 22 amino acids in length, about 23 amino acids in length, about 24 amino acids in length, about 25 amino acids in length, about 26 amino acids in length, about 27 amino acids in length, about 28 amino acids in length, about 29 amino acids in length, or about 30 amino acids in length.

The machine-learning platform predicts the likelihood that one or more tumor-specific neoantigens are immunogenic (e.g., will elicit an immune response).

Immunogenic tumor-specific neoantigens are not expressed in normal tissues. They can be presented by antigen-presenting cells to CD4+ and CD8+ T-cells to generate an immune response. In embodiments, an immune response in the subject elicited by the one or more tumor-specific neoantigens comprises presentation of the one or more tumor-specific neoantigens to the tumor cell surface. More specifically, the immune response in the subject elicited by the one or more tumor-specific neoantigens comprises presentation of the one or more tumor-specific neoantigens by one or more MHC molecules on the tumor cell. It is expected that the immune response elicited by the one or more tumor-specific neoantigens is a T-cell mediated response. The immune response in the subject elicited by the one or more tumor-specific neoantigens may involve one or more tumor-specific neoantigens being capable of presentation to T-cells by antigen presenting cells, such as dendritic cells. Preferably, the one or more tumor-specific neoantigens is capable of activating CD8+ T-cells and/or CD4+ T-cells.

In embodiments, the machine-learning platform can predict the likelihood the one or more tumor-specific neoantigens will activate CD8+ T cells. In embodiments, the machine learning platform can predict the likelihood that the one or more tumor-specific neoantigens will activate CD4+ T cells. In some instances, the machine-learning platform can predict the antibody titer that the one or more tumor-specific neoantigens can elicit. In other instances, the machine-learning platform can predict the frequency of CD8+ activation by the one or more tumor-specific neoantigens.

The machine-learning platform can include a model trained on training data. Training data can be obtained from a series of distinct subjects. The training data can comprise data derived from healthy subjects, as well as subjects having cancer. The training data may include various data that can be used to generate a probability score that indicates whether the one or more tumor-specific neoantigens will elicit an immune response in a subject. Exemplary training data can include data representing nucleotide or polypeptide sequences derived from normal tissue and/or cells, data representing nucleotide or polypeptide sequences derived from tumor tissue, data representing MHC peptidome sequences from normal and tumor tissue, peptide-MHC binding affinity measurement, or combinations thereof The reference data can further comprise mass spectrometry data, DNA sequencing data, RNA sequencing data, clinical data from healthy subjects and subjects having cancer, cytokine profiling data, T cell cytotoxicity assay data, peptide-MHC mono-or-multimer data, and proteomics data for single-allele cell lines engineered to express a predetermined MHC allele that are subsequently exposed to synthetic protein, normal and tumor human cell lines, fresh and frozen primary samples, and T-cell assays.

The machine-learning platform can be a supervised learning platform, an unsupervised learning platform, or a semi-supervised learning platform. The machine-learning platform can use sequence-based approach to generate a numerical probability that the one or more tumor-specific neoantigens can elicit an immune response (e.g., will induce a high or low antibody response or CD8+response). Sequence based predictions can include supervised machine-learning modules including, artificial neural networks (e.g., deep or otherwise), support vector machines, K-nearest neighbor, Logistic Multiple Network-constrained Regression (LogMiNeR), regression tree, random forest, adaboost, XGBoost, or hidden Markov models. These platforms require training data sets that include known MHC binding peptides.

Numerous prediction programs have been employed to predict whether a tumor-specific neoantigen can be presented on an MHC molecule and elicit an immune response. Exemplary predictive programs include, for example, HLAminer (Warren et al., Genome Med., 4:95 (2012); HLA type predicted by orienting the assembly of shotgun sequence data and comparing it with the reference allele sequence database), VariantEffect Predictor Tool (McLaren et al., Genome Biol., 17:122 (2016)), NetMHCpan (Andreatta et al., Bioinformatics., 32:511-517 (2016); sequence comparison method based on artificial neural network, and predict the affinity of peptide-MHC-I type molecular), UCSC browser (Kent et al., Genome Res., 12:996-1006 (2002)), CloudNeo pipeline (Bais et al., Bioinformatics, 33:3110-2 (2017)), OptiType (Szolek et al., Bioinformatics, 30:3310-316 (2014)), ATHLATES (Liu C et al., Nucleic Acids Res. 41:e142 (2013)), pVAC-Seq (Handal et al., Genome Med. 8:11 (2016), MuPeXI (Bjerregaard et al., Cancer Immunol Immunother., 66:1123-30 (2017)), Strelka (Saunders et al., Bioinformatics. 28:1811-7 (2012)), Strelka2 (Kim et al., Nat Methods. 2018;15:591-4.), VarScan2 (Koboldt et al., Genome Res., 22:568-76 (2012)), Somaticseq (Fang L et al., Genome Biol., 16:197 (2015)), SMMPMBEC (Kim et al., BMC Bioinformatics., 10:394 (2009)), NeoPredPipe (Schenck RO, BMC Bioinformatics., 20:264 (2019)), Weka (Witten et al., Data mining: practical machine-learning tools and techniques. 4^{th} ed. Elsevier, ISBN: 97801280435578 (eBook) (2017), or Orange (Demsar et al., Orange: Data Mining Toolbox in Python., J. Mach Learn Res., 14:2349-2353 (2013). Any known predictive programs may be employed as the machine-learning platform to generate a numerical probability score that indicates whether the neoantigen will elicit an immune response.

Depending on the machine-learning platform employed, additional filters can be applied to prioritize tumor-specific neoantigen candidates, including: elimination of hypothetical (Riken) proteins; use of an antigen processing algorithm to eliminate epitopes that are not likely to be proteolytically produced by the constitutive- or immune-proteasome and prioritization of neoantigens where the neoantigen has a higher predicted binding affinity than the corresponding wildtype sequence.

The numerical probability score can be a number between 0 and 1. In embodiments, the numerical probability score can be a number of 0, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, or 1. A tumor-specific neoantigen with a higher numerical probability score relative to a lower numerical probability score indicates that the tumor-specific neoantigen will elicit a greater immune response in the subject, and thus is likely to be a suitable candidate for an immunogenic composition. For example, a tumor-specific neoantigen with a numerical probability score of 1 will likely elicit a greater immune response in a subject than a tumor-specific neoantigen having a numerical probability score of 0.05. Similarly, a tumor-specific neoantigen having a numerical probability score of 0.5 will likely elicit a greater immune response in a subject than a tumor-specific neoantigen with a numerical probability score of 0.1.

A higher numerical probability score relative to a lower numerical probability score is preferable. Preferably, tumor-specific neoantigen having a numerical probability score of at least 0.8, 0.81, 0.82. 0.83, 0.84, 0.85, 0.86, 0.87, 0.88, 0.89, 0.9, 0.95, 0.96, 0.97, 0.98, 0.99, or 1 indicates that an immune response will likely be elicited in the subject.

While a higher numerical probability score is preferable, a lower numerical probability score may still indicate that the tumor-specific neoantigen is capable of eliciting a sufficient immune response, such that the tumor-specific neoantigen is likely to be a suitable candidate.

In instances, the machine-learning platform described herein can also predict the likelihood that the one or more tumor-specific neoantigens will be presented by a MHC molecule on a tumor cell. The machine-learning platform can predict the likelihood that one or more tumor-specific neoantigens will be presented by a MHC class I molecule or MHC class II molecule.

The methods for selecting one or more tumor-specific neoantigens may further comprise a step of measuring, in silico, the affinity of one or more tumor-specific neoantigens to bind to a MHC molecule in the subject. A tumor-specific neoantigen that has a binding affinity with a MHC molecule of less than about 1000 nM indicates that the one or more tumor-specific neoantigens may be suitable for an immunogenic composition. A tumor-specific neoantigen that has a binding affinity with a MHC molecule of less than about 500 nM, of less than about 400 nM, of less than about 300 nM, of less than about 200 nM, of less than about 100 nM, of less than about 50 nM can indicate that one or more tumor-specific neoantigens may be suitable for an immunogenic composition. The affinity of the one or more tumor-specific neoantigens to bind to a MHC molecule in the subject can predict tumor-specific neoantigen immunogenicity. Alternatively, median affinity can be an effective way to predict tumor-specific neoantigen immunogenicity. Median affinity can be calculated using epitope prediction algorithms, such as NetMHCpan, ANN, SMM and SMMPMBEC.

RNA expression of one or more tumor-specific neoantigens is also quantified. RNA expression of one or more tumor-specific neoantigens is quantified to identify one or more neoantigens that will elicit an immune response in a subject. A variety of methods exist for measuring RNA expression. Known techniques, which may measure RNA expression, include RNA-seq, and in situ hybridization (e.g., FISH), Northern blot, DNA microarray, Tiling array, and quantitative polymerase chain reaction (qPCR). Other known techniques in the art can be used to quantify RNA expression. RNA can be messenger RNA (mRNA), short-interfering RNA (siRNA), microRNA (miRNA), circular RNA (circRNA), transfer RNA (tRNA), ribosomal RNA (rRNA), small nucleolar RNA (snRNA), Piwi-interacting RNA (piRNA), long non-coding RNA (long ncRNA), sub-genomic RNA (sgRNA), RNA from integrating or non-integrating viruses, or any other RNA. Preferably, mRNA expression is measured.

The methods disclosed herein can optionally comprise sequencing tumor clones. Tumor clones are sequenced to identify one or more tumor-specific neoantigens that represent a sufficient fraction of the tumor. Tumor clones can be sequenced, for example, using the sequence techniques disclosed herein and using other known sequencing technologies known by those skilled in the art.

In embodiments, a tumor-specific neoantigen that has a tumor clone fraction of at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, or at least about 30% across the tumor indicates that the tumor-specific neoantigen represents a sufficient fraction of the tumor. A sufficient fraction of the tumor indicates that the tumor-specific neoantigen provides sufficient genetic diversity across the tumor.

The method can further comprise measuring the ability of the one or more tumor-specific neoantigen to induce an autoimmune response in normal tissues. It is expected that a tumor-specific neoantigen that has similar sequence to a normal antigen may induce an autoimmune response in normal tissue. For example, a tumor-specific neoantigen that is at least 90%, at least 91%, at least 92%, at least 93%, at least 94%, at least 95%, at least 96%, at least 97%, at least 98%, or at least 99% similar to a normal antigen may induce an autoimmune response. Tumor-specific neoantigens that are predicted to induce an autoimmune response are not prioritized for the immunogenic composition. Tumor-specific neoantigens that are predicted to induce an autoimmune response are typically not selected for the immunogenic composition. The method can further comprise measuring the ability of the one or more tumor-specific neoantigen to invoke immunological tolerance. Tumor-specific neoantigens that are predicted to invoke immunological tolerance are not prioritized for the immunogenic composition. Tumor-specific neoantigens that are predicted to invoke immunological tolerance are not prioritized for the immunogenic composition.

A tumor-specific score is calculated based on the data generated by obtaining a numerical probability score that the one or more tumor-specific neoantigens will elicit an immune response in the subject and the RNA expression levels of the one or more tumor-specific neoantigens. The tumor clone fraction across the tumor can optionally be included in addition to the above calculation used to calculate the tumor-specific score. A tumor-specific neoantigen that has a high numerical probability score (e.g., the tumor-specific neoantigen is immunogenic) and has a high level of RNA expression, will be prioritized. In comparison, a tumor-specific antigen that is predicted to induce an autoimmune response will have a lower tumor-specific neoantigen score relative to a tumor-specific neoantigen that does not induce an immune response and will not be selected for inclusion in an immunogenic composition. A tumor-specific neoantigen that has a high numerical probability score (e.g., the tumor-specific neoantigen is immunogenic), has a high level of RNA expression, and provides a sufficient tumor clone fraction across the tumor will be prioritized.

A tumor-specific neoantigen that has a high numerical probability score (e.g., the tumor-specific neoantigen is immunogenic) and optionally provides a sufficient tumor clone fraction across the tumor, but has low levels of RNA expression, will have a lower tumor-specific score in comparison to a tumor-specific neoantigen that has a high numerical probability score, high RNA expression levels, and optionally provides sufficient tumor clone fraction across the tumor. In this example, the tumor-specific neoantigen with a lower tumor-specific score will not be prioritized over the tumor-specific neoantigen with the higher tumor-specific score. A tumor-specific neoantigen that has a high numerical probability score (e.g., the tumor-specific neoantigen is immunogenic) and has sufficient levels of RNA expression to elicit an immune response, but does not provide a sufficient tumor clone fraction across the tumor will have a lower tumor-specific score in comparison to a tumor-specific neoantigen that has a high numerical probability score, high RNA expression levels, and provides sufficient tumor clone fraction across the tumor. A tumor-specific neoantigen that has sufficient levels of RNA expression to elicit an immune response, provides a sufficient tumor fraction across the tumor, but has a low numerical probability score, will have a lower tumor-specific score in comparison to a tumor-specific neoantigen that has a high numerical probability score, high RNA expression levels, and provides a sufficient tumor fraction across the tumor.

Finally, one or more tumor-specific neoantigens based on the tumor-specific score are selected for formulation of a subject-specific immunogenic composition. In embodiments, at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 50 or more tumor-specific neoantigens are selected for the immunogenic composition. Typically, at least about 10 tumor-specific neoantigens are selected. In other instances, at least about 20 tumor-specific neoantigens are selected.

II. Methods of Treating

This disclosure also relates to methods of treating cancer in a subject in need thereof comprising administering a personalized immunogenic composition comprising one or more tumor specific neoantigens selected using the methods described herein.

The cancer can be any solid tumor or any hematological tumor. The methods disclosed herein are preferably suited for solid tumors. The tumor can be a primary tumor (e.g., a tumor that is at the original site where the tumor first arose). Solid tumors can include, but are not limited to, breast cancer tumors, ovarian cancer tumors, prostate cancer tumors, lung cancer tumors, kidney cancer tumors, gastric cancer tumors, testicular cancer tumors, head and neck cancer tumors, pancreatic cancer tumors, brain cancer tumors, and melanoma tumors. Hematological tumors can include, but are not limited to, tumors from lymphomas (e.g., B cell lymphomas) and leukemias (e.g., acute myelogenous leukemia, chronic myelogenous leukemia, chronic lymphocytic leukemia, and T cell lymphocytic leukemia).

The methods disclosed herein can be used for any suitable cancerous tumor, including hematological malignancy, solid tumors, sarcomas, carcinomas, and other solid and non-solid tumors. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. Preferably, the cancer is melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Melanoma is of particular interest. Breast cancer, lung cancer, and bladder cancer are also of particular interest.

Immunogenic compositions stimulate a subject's immune system, especially the response of specific CD8+ T cells or CD4+ T cells. Interferon gamma produced by CD8+ and T helper CD4+ cells regulate the expression of PD-L1. PD-L1 expression in tumor cells is upregulated when attacked by T cells. Therefore, tumor vaccines may induce the production of specific T cells and simultaneously upregulate the expression of PD-L1, which may limit the efficacy of the immunogenic composition. In addition, while the immune system is activated, the expression of T cell surface reporter CTLA-4 is correspondingly increased, which binds with the ligand B7-1/B7-2 on antigen-presenting cells and plays an immunosuppressant effect. Thus, in some instances, the subject may further be administered an anti-immunosuppressive or immunostimulatory, such as a checkpoint inhibitor. Checkpoint inhibitors can include, but are not limited to, anti-CTL4-A antibodies, anti-PD-1 antibodies and anti-PD-L1 antibodies. These checkpoint inhibitors bind to the immune checkpoint proteins of T cells to remove the inhibition of T cell function by tumor cells. Blockade of CTLA-4 or PD-L1 by antibodies can enhance the immune response to cancerous cells in the patient. CTLA-4 has been shown effective when following a vaccination protocol.

An immunogenic composition comprising one or more tumor-specific neoantigens can be administered to a subject that has been diagnosed with cancer, is already suffering from cancer, has recurrent cancer (i.e., relapse), or is at risk of developing cancer. An immunogenic composition comprising one or more tumor-specific neoantigens can be administered to a subject that is resistant to other forms of cancer treatment (e.g., chemotherapy, immunotherapy, or radiation). An immunogenic composition comprising one or more tumor-specific neoantigens can be administered to the subject prior to other standard of care cancer therapies (e.g., chemotherapy, immunotherapy, or radiation). An immunogenic composition comprising one or more tumor-specific neoantigens can be administered to the subject concurrently, after, or in combination to other standard of care cancer therapies (e.g., chemotherapy, immunotherapy, or radiation).

The subject can be a human, dog, cat, horse, or any animal for which a tumor specific response is desired.

The immunogenic composition is administered to the subject in an amount sufficient to elicit an immune response to the tumor-specific neoantigen and to destroy, or at least partially arrest, symptoms and/or complications. In embodiments, the immunogenic composition can provide a long-lasting immune response. A long-lasting immune response can be established by administering a boosting dose of the immunogenic composition to the subject. The immune response to the immunogenic composition can be extended by administering to the subject a boosting dose. In embodiments, at least one, at least two, at least three or more boosting doses can be administered to abate the cancer. A first boosting dose may increase the immune response by at least 50%, at least 100%, at least 200%, at least 300%, at least 400%, at least 500%, or at least 1000%. A second boosting dose may increase the immune response by at least 50%, at least 100%, at least 200%, at least 300%, at least 400%, at least 500%, or at least 1000%. A third boosting dose may increase the immune response by at least 50%, at least 100%, at least 200%, at least 300%, at least 400%, at least 500%, or at least 1000%.

An amount adequate to elicit an immune response is defined as a “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that immunogenic compositions can generally be employed in serious disease states, that is, life-threatening or potentially life-threatening situations, especially when the cancer has metastasized. In such cases, in view of the minimization of extraneous substances and the relative nontoxic nature of a neoantigen, it is possible and can be felt desirable by the treating physician to administer substantial excesses of these immunogenic compositions.

The immunogenic composition comprising one or more tumor-specific neoantigens can be administered to the subject alone or in combination with other therapeutic agents. The therapeutic agent can be, for example, a chemotherapeutic agent, radiation, or immunotherapy. Any suitable therapeutic treatment for a particular cancer can be administered. Exemplary chemotherapeutic agents include, but are not limited to aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. The subject may be administered a small molecule, or targeted therapy (e.g. kinase inhibitor). The subject may be further administered an anti-CTLA antibody or anti-PD-1 antibody or anti-PD-Ll antibody. Blockade of CTLA-4 or PD-L1 by antibodies can enhance the immune response to cancerous cells in the patient.

III. Immunogenic Compositions

The invention further relates to personalized (i.e., subject-specific) immunogenic compositions (e.g., a cancer vaccine) comprising one or more tumor-specific antigens selected using the methods described herein. Such immunogenic compositions can be formulated according to standard procedures in the art. The immunogenic composition is capable of raising a specific immune response.

The immunogenic composition can be formulated so that the selection and number of tumor-specific neoantigens is tailored to the subject's particular cancer. For example, the selection of the tumor-specific neoantigens can be dependent on the specific type of cancer, the status of the cancer, the immune status of the subject, and the MHC-type of the subject.

The immunogenic composition can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 37, 37, 38, 39, 40, 41, 42, 43, 44, 45, 46, 47, 48, 49, 50 or more tumor-specific neoantigens. The immunogenic composition can contain about 10-20 tumor-specific neoantigens, about 10-30 tumor-specific neoantigens, about 10-40 tumor-specific neoantigens, about 10-50 tumor-specific neoantigens, about 10-60 tumor-specific neoantigens, about 10-70 tumor-specific neoantigens, about 10-80 tumor-specific neoantigens, about 10-90 tumor-specific neoantigens, or about 10-100 tumor-specific neoantigens. Preferably, the immunogenic composition comprises at least about 10 tumor-specific neoantigens. Also preferably is an immunogenic composition that comprises at least about 20 tumor-specific neoantigens.

The immunogenic composition can further comprise natural or synthetic antigens. The natural or synthetic antigens can increase the immune response. Exemplary natural or synthetic antigens include, but are not limited to, pan-DR epitope (PADRE) and tetanus toxin antigen.

The immunogenic composition can be in any form, for example a synthetic long peptide, RNA, DNA, a cell, a dendritic cell, a nucleotide sequence, a polypeptide sequence, a plasmid, or a vector.

Tumor-specific neoantigens can also be included in viral vector-based vaccine platforms, such as vaccinia, fowlpox, self-replicating alphavims, marabavirus, adenovirus (See, e.g., Tatsis et al., Molecular Therapy, 10:616-629 (2004)), or lentivirus, including but not limited to second, third or hybrid second/third generation lentivirus and recombinant lentivirus of any generation designed to target specific cell types or receptors (See, e.g., Hu et al., Immunol Rev., 239(1): 45-61 (2011), Sakma et al, Biochem J., 443(3):603-18 (2012)). Dependent on the packaging capacity of the above-mentioned viral vector-based vaccine platforms, this approach can deliver one or more nucleotide sequences that encode one or more tumor-specific neoantigen peptides. The sequences may be flanked by non-mutated sequences, may be separated by linkers or may be preceded with one or more sequences targeting a subcellular compartment (See, e.g., Gros et al., Nat Med., 22 (4):433-8 (2016), Stronen et al., Science., 352(6291): 1337-1341 (2016), Lu et al., Clin Cancer Res., 20(13):3401-3410 (2014)). Upon introduction into a host, infected cells express the one or more tumor-specific neoantigens, and thereby elicit a host immune (e.g., CD8+ or CD4+) response against the one or more tumor-specific neoantigens. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of neoantigens that will be apparent to those skilled in the art from the description herein may also be used.

The immunogenic composition can contain individualized components, according to their personal needs of the particular subject.

The immunogenic composition described herein can further comprise an adjuvant. Adjuvants are any substance whose admixture into an immunogenic composition increases, or otherwise enhances and/or boosts, the immune response to a tumor-specific neoantigen, but when the substance is administered alone does not generate an immune response to a tumor-specific neoantigen. The adjuvant preferably generates an immune response to the neoantigen and does not produce an allergy or other adverse reaction. It is contemplated herein that the immunogenic composition can be administered before, together, concomitantly with, or after administration of the immunogenic composition.

Adjuvants can enhance an immune response by several mechanisms including, e.g., lymphocyte recruitment, stimulation of B and/or T cells, and stimulation of macrophages. When an immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see, GB 2220211), MF59 (Novartis), AS03 (Glaxo SmithKline), AS04 (Glaxo SmithKline), polysorbate 80 (Tween 80; ICL Americas, Inc.), imidazopyridine compounds (see, International Application No. PCT/US2007/064857, published as International Publication No. WO2007/109812), imidazoquinoxaline compounds (see, International Application No. PCT/US2007/064858, published as International Publication No. WO2007/109813) and saponins, such as QS21 (see, Kensil et al, in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see, Stoute et al, N. Engl. J. Med. 336, 86-91 (1997)).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:Cl2U), poly ICLC, non-CpG bacterial DNA or RNA as well as immunoactive small molecules and antibodies such as cyclophosphamide, sunitmib, bevacizumab, Celebrex (celecoxib), NCX-4016, sildenafil, tadalafil, vardenafil, sorafinib, XL-999, CP-547632, pazopamb, ZD2171, AZD2171, ipilimumab, tremelimumab, and SC58175, which may act therapeutically and/or as an adjuvant. In embodiments, Poly ICLC is a preferable adjuvant.

The immunogenic compositions can comprise one or more tumor-specific neoantigens described herein alone or together with a pharmaceutically acceptable carrier. Suspensions or dispersions of one or more tumor-specific neoantigens, especially isotonic aqueous suspensions, dispersions, or ampgipgilic solvents can be used. The immunogenic compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

An excipient can be present independently of an adjuvant. The function of an excipient can be, for example, to increase the molecular weight of the immunogenic composition, to increase activity or immunogenicity, to confer stability, to increase the biological activity, or to increase serum-half life. An excipient can also be used to aid presentation of the one or more tumor-specific neoantigens to T-cells (e.g., CD 4+ or CD8+ T-cells). The excipient can be a carrier protein such as, but not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. Alternatively, the carrier can be dextran, for example sepharose.

Cytotoxic T-cells recognizes an antigen in the form of a peptide bound to an MHC molecule, rather than the intact foreign antigen itself. The MHC molecule itself is located at the cell surface of an antigen presenting cell. Thus, an activation of cytotoxic T-cells is possible if a trimeric complex of peptide antigen, MHC molecule, and antigen-presenting cell (APC) is present. It may enhance the immune response if not only the one or more tumor-specific antigens are used for activation of cytotoxic T-cells, but if additional APCs with the respective MHC molecule are added. Therefore, in some embodiments an immunogenic composition additionally contains at least one APC.

The immunogenic composition can comprise an acceptable carrier (e.g., an aqueous carrier). A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Neoantigens can also be administered via liposomes, which target them to a particular cell tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the neoantigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired neoantigen can be directed to the site of lymphoid cells, where the liposomes then deliver the selected immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., An. Rev. Biophys. Bioeng. 9;467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

An alternative method for targeting immune cells, components of the immunogenic composition, such as an antigen (i.e., tumor-specific neoantigen), ligand, or adjuvant (e.g., TLR) can be incorporated into an poly(lactic-co-glycolic) microspheres. The poly(lactic-co-glycolic) microspheres can entrap components of the immunogenic composition as an endosomal delivery device.

For therapeutic or immunization purposes, nucleic acids encoding a tumor-specific neoantigen described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990), as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation. The nucleic acids can also be delivered complexed to cationic compounds, such as cationic lipids.

The immunogenic compositions provided herein can be administered to the subject by, including but not limited to, oral, intradermal, intratumoral, intramuscular, intraperitoneal, intravenous, topical, subcutaneous, percutaneous, intranasal and inhalation routes, and via scarification (scratching through the top layers of skin, e.g., using a bifurcated needle). The immunogenic composition can be administered at the tumor site to induce a local immune response to the tumor.

The dosage of the one or more tumor-specific neoantigens may depend upon the type of composition and upon the subject's age, weight, body surface area, individual condition, the individual pharmacokinetic data, and the mode of administration.

Also disclosed herein is a method of manufacturing an immunogenic composition comprising one or more tumor-specific neoantigens selected by performing the steps of the methods disclosed herein. An immunogenic composition as described herein can be manufactured using methods known in the art. For example, a method of producing a tumor-specific neoantigen or a vector (e.g., a vector including at least one sequence encoding one or more tumor-specific neoantigens) disclosed herein can include culturing a host cell under conditions suitable for expressing the neoantigen or vector, wherein the host cell comprises at least one polynucleotide encoding the neoantigen or vector, and purifying the neoantigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes one or more tumor-specific neoantigens or vector disclosed herein. In certain embodiments the isolated polynucleotide can be cDNA.

IV. Samples

The methods disclosed herein comprise selecting one or more tumor-specific neoantigens derived from a tumor. The methods of selecting one or more tumor-specific neoantigens comprise obtaining sequence data derived from the tumor. Such sequence data can be derived from a tumor sample of a subject. The tumor sample can be obtained from a tumor biopsy.

The tumor sample can be obtained from human or non-human subjects. Preferentially, the tumor sample is obtained from a human. The tumor sample can be obtained from a variety of biological sources that comprise cancerous tumors. The tumor can be from a tumor site or circulating tumor cells from blood. Exemplary samples can include, but are not limited to, bodily fluid, tissue biopsies, blood samples, serum plasma, stool, skin samples, and the like. The source of a sample can be a solid tissue sample such as a tumor tissue biopsy. Tissue biopsy samples may be biopsies from, e.g., lung, prostate, colon, skin, breast tissue, or lymph nodes. Samples can also be e.g., samples of bone marrow, including bone marrow aspirate and bone marrow biopsies. Samples can also be liquid biopsies, e.g., circulating tumor cells, cell-free circulating tumor DNA, or exosomes. Blood samples can be whole blood, partially purified blood, or a fraction of whole or partially purified blood, such as peripheral blood mononucleated cells (PBMCs).

The tumor samples described herein can be obtained directly from a subject, derived from a subject, or derived from samples obtained from a subject, such as cultured cells derived from a biological fluid or tissue sample. The tumor biopsy can be a fresh sample. The fresh sample can be fixed after removal from the subject with any known fixatives (e.g. formalin, Zenker's fixative, or B-5 fixative). The tumor biopsy can also be archived samples, such as frozen samples, cryopreserved samples, of cells obtained directly from a subject or of cells derived from cells obtained from a subject. Preferably, the tumor sample obtained from a subject is a fresh tumor biopsy.

The tumor sample can be obtained from a subject by any means including, but not limited to, tumor biopsy, needle aspirate, scraping, surgical excision, surgical incision, venipuncture, or other means known in the art. A tumor biopsy is a preferred method for obtaining the tumor. The tumor biopsy can be obtained from any cancerous site, for example, a primary tumor or a secondary tumor. A tumor biopsy from a primary tumor is generally preferred. Those skilled in the art will recognize other suitable techniques for obtaining tumor samples.

The tumor sample can be obtained from the subject in a single procedure. The tumor sample can be obtained from the subject repeatedly over a period of time. For example, the tumor sample may be obtained once a day, once a week, monthly, biannually, or annually. Obtaining numerous samples over a period of time can be useful to identify and select new tumor-specific neoantigens. The tumor sample can be obtained from the same tumor or different tumors.

The tumor sample can be obtained from the primary tumor, one or more metastases, and/or individual sites of tumor growth (e.g., bone marrow from different skeletal parts, such as hip, bone, or vertebra). The tumor sample can be obtained from the same site or different site.

### 5. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

### 6. EXAMPLES

The following are examples of methods and compositions of the invention. It is understood that various other embodiments may be practiced, given the general description provided herein.

### Example 1. Neoantigen Peptide Selection

This example describes the individual procedural steps to select neo-antigenic, immunogenic peptides identified from next generation sequencing data generated from a patient's tumor and normal tissue.

### 1.1. Sample Preparation and Generation of WES, WGS, and RNA-Seq Data

Tumor biopsies or surgical explants were collected from study participants with informed consent and transported to the Clinical Trials CLIA laboratory in tissue culture medium on ice. There, samples were accessioned, and assigned with a specific unique sample identifier. Next, tissue was weighed, portioned, and placed in five (5) times its volume of RNAlater Stabilization Solution (ThermoFisher, CatNo AM7020). The samples were then left overnight at 4° C., removed from the RNAlater solution, and placed in a cryovial with 1 mL STEMCELL CroyStor10 (CatNo 07952) and transferred into a CoolCell (Corning, CatNo 432000) at −80° C.

Peripheral Blood collected in ACD tubes from participants in the accompanying Study Protocol, were transported to a Specimen Processing & Research Cell Bank, where PBMC processing occured according to SOP using Ficoll. PBMC processing may occur prior to tumor biopsy to allow simultaneous shipping of PBMC and tumor biopsy tissues to a sequencing provider.

TABLE 1
Samples

| | | | Tumor | Manufacturing Steps Performed | | | | |
|---|---|---|---|---|---|---|---|---|
| Sample | | | Sample | | | RNA | HLA | Peptide |
| ID | Biopsy Type | Gender | Type | WES | WGS | Seq | Haplotyping | Manufacturing |
| AAAAA | Subcutaneous | Male | Single cell | X | — | X | X | — |
| | metastasis | | suspension¹ | | | | | |
| BBBBB | Lymph node | Female | Single cell | X | — | X | X | — |
| | metastasis | | suspension¹ | | | | | |
| CCCCC | Lymph node | Male | Single cell | X | — | X | X | — |
| | metastasis | | suspension¹ | | | | | |
| DDDDD | Lymph node | Male | Single cell | X | — | X | X | — |
| | metastasis | | suspension¹ | | | | | |
| EEEEE | Lymph node | Male | Mock | X | X | X | X | X |
| | metastasis | | biopsy | | | | | |
| | | | from tumor | | | | | |
| | | | tissue | | | | | |
| | | | (156 mg | | | | | |
| | | | tissue)² | | | | | |
| FFFFF | Lymph node | Male | Single cell | X | X | X | X | X |
| | metastasis | | suspension | | | | | |
| | | | (100,000 | | | | | |
| | | | cells)¹ | | | | | |
| GGGG | Lymph node | Female | Mock | X | X | X | X | X |
| | metastasis | | biopsy | | | | | |
| | | | from tumor | | | | | |
| | | | tissue (30 | | | | | |
| | | | mg tissue)³ | | | | | |

Notes:
¹Single cell suspensions were generated through tissue digest and disruption
²Two passes with a 3 mm punch tool
³A total of 10 passes with 14 gauge needle

All specimens (tumor biopsy and PBMC) were sent by the CLIA laboratory overnight to the sequencing provide on dry ice.

DNA, RNA, and miRNA were simultaneously isolated from the same tissue or cell specimen using the AllPrep DNA/RNA/miRNA Universal Kit (QIAGEN).

DNA/RNA sample quality and quantity were assessed using adequeate methods (e.g. Qubit, BioAnalyzer) and the following metrics were recorded:

DNA: concentration (ng/μL), total amount (ng), volume (μL)

RNA: concentration (ng/μL), total amount (ng), volume (μL), purity (RIN)

TABLE 2
Genomic DNA and Total RNA Yields from Engineering Run Samples RNA

| | Amount | Concentration | Volume | Amount | Concentration | | Amount | Concentration | Volume | |
|---|---|---|---|---|---|---|---|---|---|---|
| | (ng) | (ng/mL) | (μL) | (ng) | (ng/nL) | Volume | (ng) | (ng/nL) | (μL) | RIN |
| AAAAA | 11 | 1 | 16 | 341 | 34 | 10 | 171 | 24 | 8 | 7.2 |
| BBBBB | 126 | 8 | 16 | 239 | 24 | 10 | 172 | 25 | 8 | 3.3 |
| CCCCC | 12 | 1 | 16 | 180 | 18 | 10 | 173 | 25 | 8 | 4.5 |
| DDDDD | 83 | 5 | 16 | 160 | 16 | 10 | 195 | 24 | 8 | 2.6 |
| EEEEE | 23144 | 263 | 88 | 4039 | 46 | 88 | 40035 | 702 | 57 | 6.8 |
| FFFFF | 4136 | 47 | 88 | 4391 | 50 | 88 | 1427 | 25 | 57 | 8.4 |
| GGGGG | 203 | 2 | 88 | 3467 | 39 | 88 | 44433 | 780 | 57 | 5.4 |

DNA samples containing more than 200 ng of genomic DNA were aliquoted, with 200 ng used for WES, and remaining DNA was shipped for another sequencing provider for WGS.

NGS was performed. An overview of the library preparation and sequencing strategies is show in Table 3 below.

TABLE 3
Details of Next Generation Sequencing performed in
PNV-21 manufacturing

| NGS | | Sequencing | Sequencing |
|---|---|---|---|
| Method | Target Depth | Strategy | Instrument |
| WES | 75× Normal, | 100 bp PE | NovaSeq 6000 |
| | 125× Tumor | | |
| RNA-Seq | 100M | 50 bp PE | Nova Seq 6000 |
| WGS | 250× Normal, | 101 bpPE | Nova Seq 6000 |
| | 250× Tumor | | |

WES data from paired tumor/normal samples was used to identify germline and somatic variants of the patient and her/his tumor.

WES is performed at a commercial sequencing vendor using the Agilent SureSelect All Exon v6 bait kit and libraries were generated and sequenced on an Illumina NovaSeq6000 instrument. DNA from PBMC samples underwent WES using a 100bp PE strategy, 75× average coverage, and a target of 38 million reads. DNA from tissue samples undergoes WES using a 100 bp PE strategy, 125× average coverage, and a target of 63 million reads.

RNA sequencing data was used to identify neo-antigen encoding RNA transcripts with sufficiently high expression, as well as to independently confirm WES derived somatic variants.

RNA was sequenced using the Illumina Stranded mRNA sequencing method using a 50bp PE strategy and a target of 100 million reads.

Sequencing libraries were created using the Illumina TruSeq Stranded mRNA method, preferentially selecting for messenger RNA by taking advantage of the polyadenylated tail.

WGS data was used to perform CNV calling and to identify subclones of the tumor samples.

WGS was performed at a commercial, CLIA validated laboratory from tumor and normal genomic DNA prepared as detailed above. Two pooled libraries from the same individual were sequenced on an Illumina S4 flow cell (FC) with read length of 2×101. Data generated on an FC with Q30>80% and error rate <3% was passed for demultiplexing.

NGS data in FASTQ format was transferred from the sequencing vendors for further bioinformatics analysis.

HLA typing was performed with a molecular assay at an accredited clinical immunogenetics laboratory.

### 1.2. Bioinformatics Analysis of NGS Data

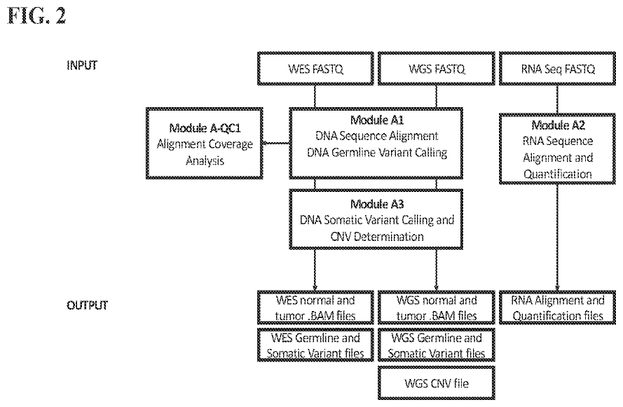

Mapping and Alignment of NGS reads to the hg19 reference genome was done using the Illumina DRAGEN Bio-IT Platform (v3.8.5). Details about the input and output files, as well as the automated execution of the processing steps used for mapping and alignment are described in FIG. 2

All NGS analyses were performed using the hg19 human reference genome assembly (initial UCSC reference assembly, based on initial GRCh37 release, md5 checksum a244d8a32473650b25c6e8e1654387d6, downloaded from the Sentieon reference bundle. Mapping between known ENSEMBL genes and hg19 chromosomal coordinates (GTF file) was downloaded from UCSC. This file is used for quantification of RNA gene expression. The DRAGEN Bio-IT platform was used to generate a series of hash-table files, which are needed for mapping, alignment, and variant calling. The generated hash table files and the hg19 ENSEMBLE GTF file were uploaded to an S3 data storage bucket.

The DNA sequence mapping and alignment step took NGS FASTQ files as input and aligns reads to the provided reference genome hash tables, independently for normal and tumor (if provided) samples. Normal mapping/alignment included the generation of germline variant calls, used in Module A3 for somatic CNV calling (termed B-Allele file).

TABLE 4
NGS FASTQ input

| | |
|---|---|
| Module A1 | DNA Seq Mapping/Alignment-- |
| | NORMAL |

| | |
|---|---|
| Environment | Version |
| Illumina DRA GEN Bio-IT Platform | 3.8.5 |

```
Command Line
dragen -f -r $BUNDLE_FOLDER −1 $NORMAL_FASTQ 1 −2 $NORMAL_FASTQ2 \
  --RGID “${NORMAL_GROUP}” --RGSM “${NORMAL_SAMPLE}” \
  --RGPL ILLUMINA \
  --dupmark-version sort \
  --enable-variant-caller true \
  --enable-map-align-output true \
  --enable-vcf-compression true \
  --intermediate-results-dir ${TMP_FOLDER} \
  --enable-bam-indexing true \
  --output-directory ${ANALYSIS_FOLDER}/ \
  --output-file-prefix ${SM_Prefix} \
  --lic-server ${LICENSE_STRING}
Input
```

| Variable | Value | Description |
|---|---|---|
| -f | | Force overwrite |
| -r | $BUNDLE_FOLDER | Location of Hash Table folder |
| −1, −2 | $NORMAL_FASTQ1, | Location of normal FASTQ files |
| | $NORMAL_FASTQ2 | |
| --RGID | ${NORMAL_GROUP} | Read group ID string, normal |
| --RGSM | ${NORMAL_SAMPLE} | Read group Sample Name string, normal |
| --RGPL | ILLUMINA | Read group platform string, normal |
| --dupmark-version | sort | Enable memory optimized germline calling |
| --enable-variant-caller | true | Enable variant calling |
| --enable-map-align- | true | Output .BAM file |
| output | | |
| --enable-vcf- | true | Enable vcf compression |
| compression | | |
| --intermediate-results- | ${TMP_FOLDER} | Temporary files directory, scratch |
| dir | | |
| --enable-bam- | true | Enable indexing of BAM files |
| indexing | | |
| --output-directory | ${ANALYSIS_FOLDER} | Local directory for file output |
| --output-file-prefix | ${SM_Prefix} | Prefix for output files, sample name |
| --lic-server | ${LICENSE_STRING} | String with |
| | | User:Password@license.edicogenome.com |

```
Output
A number of files were generated inside the output directory, all prefixed with ${SM_Prefix}:
  • Normal BAM file (plus index)
  • Normal VCF file (plus index), unfiltered and “hard-filtered”
  • Mapping quality metrics report
  • FastQC report
```

In addition to the mapping/alignment algorithm, this module utilized DRAGEN's reporting function (including mapping statistics and trimming report), which was used for quality control.

For tumor sample mapping/alignment, the command line was outlined in Table 5.

TABLE 5
Comand line for tumor sample mapping/alignment

| | |
|---|---|
| Module A1 | DNA Seq Mapping/Alignment-- |
| | TUMOR |

| | |
|---|---|
| Environment | Version |
| Illumina DRA GEN Bio-IT Platform | 3.8.5 |

```
Command Line
dragen -f-r $BUNDLE_FOLDER −1 $TUMOR_FASTQ1 −2 $TUMOR_FASTQ2 \
  --enable-variant-caller false \
  --RGID “${TUMOR_GROUP}” --RGSM “${TUMOR_SAMPLE}” \
  --RGPL ILLUMINA \
  --dupmark-version sort \
  --enable-map-align-output true \
  --enable-vcf-compression true \
  --intermediate-results-dir ${TMP_FOLDER} \
  --enable-bam-indexing true \
  --output-directory ${ANALYSIS_FOLDER}/ \
  --output-file-prefix ${SM_Prefix} tumor \
  --lic-server ${LICENSE_STRING}
Input
```

| Variable | Value | Description |
|---|---|---|
| -f | | Force overwrite |
| -r | SBUNDLE_FOLDER | Location of Hash Table folder |
| −1, −2 | STUMOR_FASTQ1, | Location of tumor FASTQ files |
| | STUMOR_FASTQ2 | |
| --RGID | ${TUMOR_GROUP} | Read group ID string, normal |
| --RGSM | ${TUMOR_SAMPLE} | Read group Sample Name string, normal |
| --RGPL | ILLUMINA | Read group platform string, normal |
| --enable-variant-caller | true | Enable variant calling |
| --dupmark-version | sort | Enable memory optimized germline calling |
| --enable-map-align- | true | Output. BAM file |
| output | | |
| --enable-vcf- | true | Enable vcf compression |
| compression | | |
| --intermediate-results- | ${TMP_FOLDER} | Temporary files directory, scratch |
| dir | | |
| --enable-bam- | true | Enable indexing of BAM files |
| indexing | | |
| --output-directory | ${ANALYSIS_FOLDER} | Local directory for file output |
| --output-file-prefix | ${SM_Prefix} | Prefix for output files, sample name |
| --lic-server | ${LICENSE_STRING} | String with |
| | | User:Password@license.edicogenome.com |

```
Output
A number of files were generated inside the output directory, all prefixed with ${SM_Prefix}:
  • Tumor BAM file (plus index)
  • Mapping quality metrics report
  • FastQC report
```

RNA Seq FASTQ files were aligned to the UCSC hg19 human reference genome using the DRAGEN Bio-IT Platform (v3.8.5) RNA module with quantification and gene fusion detection enabled. A GENCODE hg19/GRCg37.p13 GTF file was used to map genes and gene transcripts (Ensembl gene and transcript IDs) to genomic regions.

TABLE 6
Comand line for RNA Sequence Alignment and RNA Quantification

| | |
|---|---|
| Module A2 | RNA Seq Alignment and RNA Quantification |

| | |
|---|---|
| Environment | Version |
| Illumina DRA GEN Bio-IT Platform | 3.8.5 |

```
Command Line
dragen -f -r $BUNDLE_FOLDER \
  -a ${USE_BUNDLE_GFF} \
  −1 $RNA_FASTQ1 −2 $RNA_FASTQ2 \
  --enable-map-align-output true \
  --enable-rna true V
  --enable-rna-quantification true \
  --enable-rna-gene-fusion true \
  --enable-bam-indexing true \
  --enable-variant-caller false \
  --RGID “${RNA_GROUP}” \
  --RGSM “${RNA_SAMPLE}” \
  --output-directory ${ANALYSIS_FOLDER}/ \
  --output-file-prefix ${SM_Prefix} RNA \
  --intermediate-results-dir ${IMP_FOLDER} \
  --lic-server ${LICENSE_STRING}
Input
```

| Variable | Value | Description |
|---|---|---|
| -f | | Force overwrite |
| -r | $BUNDLE_FOLDER | Location of Hash Table folder |
| -a | ${USE_BUNDLE_GFF} | Location of .GFF/GTF file |
| −1, −2 | $RNA_FASTQ1, | Location of RNA Seq FASTQ files |
| | $RNA_FASTQ2 | |
| --enable-map-align-output | true | Enable mapping/alignment |
| --enable-rna | true | Enable RNA processing |
| --enable-rna-quantification | true | Enable quantification of gene expression |
| --enable-rna-gene-fusion | true | Enable detection of gene fusions |
| --enable-bam-indexing | true | Enable indexing of .BAM file |
| --enable-variant-caller | False | Disable Variant Calling |
| --RGID | ${RNA_GROUP} | Read Group ID |
| --RGSM | ${RNA_SAMPLE} | Read Group Sample Name |
| --output-directory | ${ANALYSIS_FOLDER}/ | Output folder for result files |
| --output-file-prefix | ${SM_Prefix}_RNA | Prefix for output files, SM_Prefix is sample |
| | | name |
| --intermediate-re suits-dir | ${TMP_FOLDER} | Temporary files directory, scratch |
| --lic-server | ${LICENSE_STRING} | String with |
| | | User:Password@license.edicogenome.com |

```
Output
A number of files were generated inside the output directory, all prefixed with ${SM_Prefix}_RNA:
  • RNA BAM file
  • RNA gene expression and transcript quantification files ${SM_Prefix}_RNA. quant.genes.sf and
    ${SM Prefix} RNA.quant.sf, respectively.
  • read trimming report
```

Normal (PBMC) WES and WGS.BAM files, aligned to the UCSC hg19 human reference genome, were used individually to generate a list (.VCF file) of germline variant calls, indicating differences in the study participant's genome versus the reference genome. Variants detected were single or multiple base mutations, insertions, and deletions. Structural variants were not processed. The detailed command line argument to derive germline variant calls and produce a non-filtered, and filtered germline .VCF file with the DRAGEN Bio-IT Platform (v3.8.5) is described below.

The resultant germline .VCF file was used for ensuring that candidate vaccine peptides are not a representation of the study participant's germline sequence (self-peptides), and further serves CNV calling as input B-allele frequency file.

Somatic variant occurring as differences between tumor and normal study participant samples were identified from WES and WGS aligned .BAM files using the DRAGEN Bio-IT Platform. In the first step, tumor and normal .BAM files were compared to identify tumor specific (somatic) DNA mutations, which were output as .VCF files, one non-filtered, and one filtered for high confidence variants. In a second step, and for WGS only, CNV calls were produced and output as .VCF file. Input for this step were tumor and normal .BAM files, as well as the hard-filtered germline variant call file, used as B-allele frequency input.

Additional details and command line interface options for executing the somatic variant and CNV calling are described in Table 8.

TABLE 7
Automated workflow for DNA somatic variant calling

| | |
|---|---|
| Module A3 | DNA Somatic Variant Calling |

| | |
|---|---|
| Environment | Version |
| Illumina DRA GEN Bio-IT Platform | 3.8.5 |

```
Command Line
  dragen -v \
    -f\
    -r $BUNDLE_FOLDER \
    --intermediate-results-dir $TMP_FOLDER \
    --enable-cnv false \
    --enable-map-align false \
    --enable-variant-caller true \
    --dupmark-version sort \
    --enable-vcf-compression true \
    --bam-input ${normal_input} \
    --tumor-bam-input ${tumor_input} \
    --output-directory ${ANALYSIS_FOLDER}/ \
    --output-file-prefix ${SM_Prefix}-TN \
    --lic-server ${LICENSE_STRING} \
    $BED_OPTION
Input
```

| Variable | Value | |
|---|---|---|
| -f | | Force overwrite |
| -r | $BUNDLE_FOLDER | Location of Hash Table folder |
| -v | | Verbose output |
| --intermediate- | ${TMP_FOLDER} | Temporary files directory, scratch |
| results-dir | | |
| --enable-cnv | false | Disable CNV calling. |
| --enable-map-align | false | Disable map/align step |
| --enable-variant- | true | Enable variant calling |
| caller | | |
| --dupmark-version | sort | Enable memory optimized germline calling |
| --enable-vcf- | true | Enable vcf compression |
| compression | | |
| --bam-input | ${normal_input} | Normal .BAM output from Module A2 |
| --tumor-bam-input | ${tumor_input} | Tumor .BAM output from Module A2 |
| --output-directory | ${ANALYSIS_FOLDER}/ | Output folder for result files |
| --output-file-prefix | ${SM_Prefix}-TN | Prefix for output files, SM_Prefix is sample name |
| --lic-server | ${LICENSE_STRING} | String with |
| | | User:Password@license.edicogenome.com |
| Optional | | |
| $BED_OPTION | -vc-target-bed ${BEDFILE} | Use a .BED file to restrict processing. |

```
Output
A number of files were generated inside the output directory, all prefixed with ${SM_Prefix}-TN:
  • Somatic .VCF file
  • Somatic .VCF file with default filters applied, ${SM_Prefix}-TN-hard.filtered.vcf
  • CNV calling .VCF, .GFF3 files
  • Additional analytics files
```

The somatic .VCF file (hard-filtered) were used for downstream vaccine peptide selection modules.

CNV calling in DRAGEN requires a B-Allele .VCF file. CNV calling was preceded by germline calling and normal/tumor .BAM files from Module A1.

TABLE 8
Module A4 DNASomatic CNV Calling

| | |
|---|---|
| Module A3 | DNA Somatic CNV Calling |

| | |
|---|---|
| Environment | Version |
| Illumina DRA GEN Bio-IT Platform | 3.8.5 |

```
Command Line
  dragen -v \
    -f \
    -r $BUNDLE_FOLDER \
    --intermediate-results-dir $TMP_FOLDER \
    --enable-cnv true \
    --cnv-enable-plots true \
    --cnv-normal-b-allele-vcf ${USE_DRAGENgermline_hf_vcf} \
    --enable-map-align false \
    --enable-variant-caller false \
    --bam-input ${normal_input} \
    --tumor-bam-input ${tumor_input} \
    --output-directory ${ANALYSIS_FOLDER}/ \
    --output-file-prefix ${SM_Prefix}-TN \
    --lic-server ${LICENSE_STRING} \
    $BED_OPTION
Input
```

| Variable | Value | Description |
|---|---|---|
| -f | | Force overwrite |
| -r | $BUNDLE_FOLDER | Location of Hash Table folder |
| -v | | Verbose output |
| --intermediate- | ${TMP_FOLDER} | Temporary files directory, scratch |
| results-dir | | |
| --enable-cnv | true | Enable CNV calling. |
| --cnv-enable- | true | Enable plot output |
| plots | | |
| --cnv-normal-b- | ${USE_DRAGENgermline_hf_vcf} | B-Allele file; Location of the normal germline |
| allele-vcf | | VCF file (hard-filtered) |
| --enable-map- | false | Disable map/align step |
| align | | |
| --enable-variant- | false | Disable variant calling |
| caller | | |
| --bam-input | ${normal_input} | Normal .BAM output from Module A2 |
| --tumor-bam- | ${tumor_input} | Tumor .BAM output from Module A2 |
| input | | |
| --output-directory | ${ANALYSIS_FOLDER}/ | Output folder for result files |
| --output-file- | ${SM_Prefix}-TN | Prefix for output files, SM_Prefix is sample |
| prefix | | name |
| --lic-server | ${LICENSE_STRING} | String with |
| | | User:Password@license.edicogenome.com |
| Optional | | |
| $BED_OPTION | -vc-target-bed ${BEDFILE} | Use a .BED file to restrict processing. |

```
Output
A number of files will be generated inside the output directory, all prefixed with ${SM_Prefix}-TN:
  • CNV calling .VCF, .GFF3 files
  • Additional analytics files
```

In order to analyze mapping/alignment efficacy across contigs, Module A-QC1 employed the mosdepth analysis program taking a .BAM file as input. For WES, additionally, a .BED file to restrict analysis to defined genomic regions was provided. The hg19 bed file for the Agilent Sure Select A11 Exon v6 capture bait set was download from the Agilent website and stored in S3 for automated download by the processing pipeline.

TABLE 9
Module A-QC1 Alignment Coverage Analysis

| | |
|---|---|
| Module A-QC1 | Alignment Coverage Analysis |

| | |
|---|---|
| Environment | Version |
| mosdepth (see: https://github.com/btentp/mosdepth) | 0.3.1 |

Command Line
mosdepth -t 4 -n -x -q 1000 ${mosdepth_bedoption} ${ANALYSIS_FOLDER}/S{SM_Prefix}_tumor
$USE_TUMOR_BAM
Input

| Variable | Value | Description |
|---|---|---|
| Positional 1 | ${ANALYSIS_FOLDER}/${SM_Prefix} tumor | Output file location |
| | | and prefix (here shown |
| | | for tumor BAM |
| | | analysis) |
| | | ANALYSIS FOLDER |
| | | is the local output |
| | | folder |
| | | SM_prefix is the |
| | | sample name prefix |
| Positional 2 | $USE_TUMOR_BAM | Input BAM file (here |
| | | shown for tumor BAM |
| | | analysis) |
| -t | 4 | Number of threads |
| -n | | Do not output per base |
| | | analysis |
| -x | | Fast mode |
| -q | 1000 | Write quantized output |
| Optional: | | |
| mosdepth_bedoption | -b ${BEDFILE} | Use BED file to restrict |
| | | analysis (WES only). |

```
Output
A number of files were generated inside the output directory, all prefixed with ${SM_Prefix}_tumor
(/or_normal):
  • Summary file
  • Global distribution file
Additional output, see mosdepth manual (https://github.com/brentp/mosdepth)
```

The mosdepth summary file is a tab delimited text file which indicates the mean coverage (column mean) for all contigs present in the .BAM file (column chrom). If a .BED file was provide, the suffix “_region” denotes metrics for a .BED restricted contig. An abbreviated listing of a mosdepth summary file is shown (e.g., Table 10).

TABLE 10
Mosdepth Summary File

| chrom | length | bases | mean | min | max |
|---|---|---|---|---|---|
| chrM | 16571 | 385781 | 23.28 | 7 | 91 |
| chrM_region | 0 | 0 | 0.00 | 0 | 0 |
| chr1 | 249250621 | 974106896 | 3.91 | 0 | 4709 |
| chr1_region | 6066056 | 716175291 | 118.06 | 0 | 4709 |
| chr2 | 243199373 | 680541943 | 2.80 | 0 | 3488 |
| chr2_region | 4449345 | 484022026 | 108.79 | 0 | 1162 |
| chr3 | 198022430 | 529198467 | 2.67 | 0 | 2197 |
| chr3_region | 3462072 | 381197919 | 110.11 | 0 | 2197 |

For analysis and quality control, the tumor and normal mosdepth summary files wereused to generate specification reports.

Example Results from Mapping Aligning are shown in Tables 11 to 15 below.

TABLE 11
WES normal (PBMC) sample per contig and total alignment coverage
Normal WES per contig coverage

| contig | AAAAA | BBBBB | CCCCC | DDDDD | EEEEE | FFFFF | GGGGG |
|---|---|---|---|---|---|---|---|
| chr1 | 91.04 | 75.46 | 83.98 | 118.06 | 88.65 | 124.08 | 134.29 |
| chr2 | 84.07 | 69.79 | 77.71 | 108.79 | 78.85 | 112.96 | 124.92 |
| chr3 | 85.35 | 71.39 | 78.97 | 110.11 | 80.91 | 114.52 | 124.62 |
| chr4 | 85.64 | 73.11 | 80.79 | 114.08 | 80.04 | 113.62 | 131.06 |
| chr5 | 85.44 | 70.48 | 78.06 | 110.34 | 79.30 | 113.73 | 124.23 |
| chr6 | 87.31 | 71.82 | 79.40 | 110.67 | 80.76 | 116.11 | 125.74 |
| chr7 | 90.42 | 75.10 | 82.71 | 117.22 | 87.48 | 124.19 | 132.08 |
| chr8 | 94.21 | 75.16 | 85.69 | 123.53 | 89.09 | 128.33 | 137.32 |
| chr9 | 90.26 | 75.37 | 82.87 | 117.47 | 87.68 | 125.38 | 133.51 |
| chr10 | 84.24 | 69.72 | 76.61 | 107.61 | 79.14 | 112.51 | 123.26 |
| chr11 | 93.49 | 77.96 | 85.98 | 119.83 | 90.87 | 127.68 | 135.81 |
| chr12 | 86.84 | 71.41 | 79.31 | 110.07 | 80.74 | 115.08 | 125.06 |
| chr13 | 81.67 | 66.81 | 73.89 | 103.30 | 73.50 | 105.97 | 119.95 |
| chr14 | 87.89 | 73.11 | 81.47 | 114.51 | 83.61 | 119.02 | 128.97 |
| chr15 | 87.06 | 75.04 | 81.88 | 115.35 | 88.59 | 123.23 | 130.83 |
| chr16 | 104.70 | 88.37 | 96.08 | 138.82 | 106.57 | 150.20 | 156.55 |
| chr17 | 98.46 | 82.36 | 90.71 | 126.92 | 97.05 | 135.76 | 142.66 |
| chr18 | 81.52 | 66.73 | 73.83 | 104.19 | 74.26 | 107.36 | 119.56 |
| chr19 | 106.65 | 89.89 | 98.79 | 141.60 | 108.96 | 154.08 | 157.96 |
| chr20 | 92.79 | 78.01 | 85.05 | 120.45 | 91.93 | 129.56 | 136.70 |
| chr21 | 94.85 | 76.80 | 84.22 | 118.44 | 90.22 | 126.95 | 135.70 |
| chr22 | 99.98 | 84.02 | 91.46 | 129.71 | 102.41 | 141.08 | 145.47 |
| chrX | 51.30 | 79.83 | 46.91 | 65.47 | 48.16 | 68.42 | 143.35 |
| chrY | 67.52 | 11.87 | 60.97 | 94.91 | 60.38 | 94.90 | 22.04 |
| total | 89.44 | 75.69 | 82.25 | 115.95 | 86.30 | 122.25 | 133.61 |

TABLE 12
WES tumor sample per contig and total alignment coverage
Tumor WES per contig coverage

| contig | AAAAA | BBBBB | CCCCC | DDDDD | EEEEE | FFFFF | GGGGG |
|---|---|---|---|---|---|---|---|
| chr1 | 111.45 | 156.87 | 165.12 | 130.31 | 168.65 | 175.27 | 167.75 |
| chr2 | 96.21 | 140.58 | 134.34 | 108.12 | 121.00 | 132.45 | 194.87 |
| chr3 | 101.00 | 143.05 | 135.00 | 106.33 | 124.52 | 160.68 | 183.05 |
| chr4 | 90.88 | 145.18 | 136.25 | 117.13 | 123.96 | 157.44 | 213.40 |
| chr5 | 94.44 | 141.95 | 146.51 | 120.00 | 122.43 | 133.47 | 132.74 |
| chr6 | 101.29 | 148.37 | 141.70 | 113.56 | 132.15 | 152.03 | 151.15 |
| chr7 | 110.34 | 140.52 | 217.99 | 182.57 | 130.89 | 222.42 | 223.98 |
| chr8 | 114.25 | 170.43 | 250.80 | 214.17 | 280.41 | 152.57 | 194.43 |
| chr9 | 113.12 | 149.56 | 128.87 | 102.43 | 76.08 | 149.03 | 177.16 |
| chr10 | 95.86 | 138.91 | 126.65 | 98.06 | 120.73 | 107.09 | 92.70 |
| chr11 | 118.91 | 158.83 | 156.03 | 121.32 | 105.10 | 151.13 | 144.34 |
| chr12 | 101.13 | 148.19 | 136.95 | 108.99 | 150.62 | 135.60 | 182.38 |
| chr13 | 85.61 | 136.52 | 127.46 | 107.24 | 117.89 | 123.71 | 103.11 |
| chr14 | 104.96 | 149.85 | 144.83 | 116.11 | 128.92 | 140.17 | 95.47 |
| chr15 | 105.21 | 149.91 | 180.22 | 139.82 | 164.08 | 182.73 | 182.92 |
| chr16 | 140.71 | 173.30 | 173.31 | 138.51 | 151.85 | 179.80 | 148.28 |
| chr17 | 132.27 | 165.93 | 155.34 | 120.67 | 148.50 | 183.20 | 90.28 |
| chr18 | 84.42 | 125.96 | 136.40 | 110.64 | 110.70 | 149.78 | 188.79 |
| chr19 | 143.86 | 182.17 | 199.42 | 158.86 | 154.05 | 183.79 | 147.17 |
| chr20 | 118.41 | 153.07 | 157.90 | 124.87 | 150.00 | 194.95 | 169.70 |
| chr21 | 111.10 | 149.81 | 165.03 | 135.30 | 132.81 | 175.10 | 182.38 |
| chr22 | 135.02 | 173.50 | 186.06 | 141.07 | 145.66 | 168.84 | 135.34 |
| chrX | 58.26 | 145.37 | 79.38 | 63.49 | 73.73 | 110.56 | 197.29 |
| chrY | 65.76 | 20.79 | 84.53 | 78.93 | 88.48 | 77.81 | 23.72 |
| total | 108.84 | 152.51 | 156.13 | 125.15 | 137.69 | 158.47 | 161.36 |

TABLE 13
WGS normal (PBMC) sample per contig
and total alignment coverage
Normal WGS per contig coverage

| contig | DDDDD | FFFFF | GGGGG |
|---|---|---|---|
| chr1 | 408.85 | 335.84 | 437.51 |
| chr2 | 440.92 | 362.63 | 469.03 |
| chr3 | 440.34 | 362.47 | 467.50 |
| chr4 | 442.12 | 362.43 | 467.41 |
| chr5 | 442.23 | 362.20 | 468.21 |
| chr6 | 443.38 | 361.16 | 465.83 |
| chr7 | 438.07 | 360.01 | 465.85 |
| chr8 | 446.34 | 363.57 | 468.54 |
| chr9 | 377.33 | 311.09 | 403.67 |
| chr10 | 431.20 | 354.94 | 460.37 |
| chr11 | 435.27 | 358.04 | 464.53 |
| chr12 | 435.37 | 358.91 | 464.05 |
| chr13 | 372.30 | 306.15 | 394.59 |
| chr14 | 372.68 | 303.29 | 392.87 |
| chr15 | 359.90 | 297.52 | 383.09 |
| chr16 | 450.19 | 335.70 | 438.14 |
| chr17 | 431.80 | 354.46 | 463.96 |
| chr18 | 429.83 | 353.34 | 456.54 |
| chr19 | 424.91 | 349.23 | 459.91 |
| chr20 | 423.16 | 348.67 | 454.41 |
| chr21 | 346.86 | 283.98 | 368.69 |
| chr22 | 307.28 | 250.94 | 330.83 |
| chrX | 223.25 | 188.30 | 454.42 |
| chrY | 150.58 | 104.17 | 17.89 |
| total | 407.22 | 331.83 | 438.21 |

TABLE 14
WGS Tumor sample per contig
and total alignment coverage
Tumor WGS per contig coverage

| contig | DDDDD | FFFFF | GGGGG |
|---|---|---|---|
| chr1 | 282.48 | 273.39 | 66.93 |
| chr2 | 270.31 | 249.52 | 94.40 |
| chr3 | 267.23 | 295.22 | 95.49 |
| chr4 | 281.26 | 295.03 | 104.84 |
| chr5 | 304.11 | 249.11 | 76.37 |
| chr6 | 282.90 | 264.26 | 68.60 |
| chr7 | 427.86 | 366.02 | 95.56 |
| chr8 | 380.36 | 250.46 | 93.94 |
| chr9 | 206.64 | 215.87 | 72.53 |
| chr10 | 244.67 | 197.83 | 44.53 |
| chr11 | 276.04 | 245.76 | 71.68 |
| chr12 | 266.30 | 246.75 | 90.22 |
| chr13 | 236.75 | 210.90 | 46.36 |
| chr14 | 236.57 | 208.52 | 40.28 |
| chr15 | 271.98 | 255.83 | 68.02 |
| chr16 | 278.66 | 230.60 | 49.83 |
| chr17 | 258.87 | 275.75 | 36.02 |
| chr18 | 278.98 | 287.59 | 92.71 |
| chr19 | 296.80 | 239.13 | 48.70 |
| chr20 | 257.91 | 287.12 | 71.35 |
| chr21 | 243.95 | 227.28 | 72.47 |
| chr22 | 208.05 | 172.38 | 33.75 |
| chrX | 133.49 | 174.43 | 91.72 |
| chrY | 74.88 | 48.51 | 2.64 |
| total | 270.11 | 249.38 | 74.13 |

TABLE 15
RNA seq tumor sample per contig and total alignment coverage
tumor RNA per contig coverage

| contig | AAAAA | BBBBB | CCCCC | DDDDD | EEEEE | FFFFF | GGGGG |
|---|---|---|---|---|---|---|---|
| chr1 | 3.64 | 5.06 | 5.37 | 4.30 | 5.62 | 5.73 | 5.54 |
| chr2 | 2.43 | 3.50 | 3.39 | 2.77 | 3.11 | 3.37 | 4.96 |
| chr3 | 2.39 | 3.37 | 3.22 | 2.58 | 3.01 | 3.85 | 4.46 |
| chr4 | 1.56 | 2.43 | 2.33 | 2.02 | 2.17 | 2.72 | 3.69 |
| chr5 | 1.96 | 2.90 | 3.06 | 2.54 | 2.60 | 2.79 | 2.85 |
| chr6 | 2.43 | 3.52 | 3.41 | 2.77 | 3.17 | 3.66 | 3.67 |
| chr7 | 2.86 | 3.58 | 5.58 | 4.73 | 3.44 | 5.76 | 5.79 |
| chr8 | 2.19 | 3.31 | 4.71 | 4.04 | 5.61 | 2.98 | 3.92 |
| chr9 | 2.69 | 3.54 | 3.10 | 2.50 | 1.86 | 3.59 | 4.34 |
| chr10 | 2.43 | 3.46 | 3.21 | 2.52 | 3.10 | 2.72 | 2.38 |
| chr11 | 4.00 | 5.33 | 5.27 | 4.16 | 3.63 | 5.12 | 5.05 |
| chr12 | 3.32 | 4.80 | 4.47 | 3.61 | 5.02 | 4.46 | 6.07 |
| chr13 | 1.15 | 1.79 | 1.72 | 1.47 | 1.62 | 1.69 | 1.39 |
| chr14 | 2.61 | 3.69 | 3.63 | 2.95 | 3.26 | 3.51 | 2.43 |
| chr15 | 3.22 | 4.50 | 5.48 | 4.31 | 5.05 | 5.57 | 5.61 |
| chr16 | 5.38 | 6.61 | 6.65 | 5.37 | 5.91 | 6.89 | 5.77 |
| chr17 | 7.34 | 9.08 | 8.56 | 6.73 | 8.31 | 10.13 | 5.06 |
| chr18 | 1.46 | 2.12 | 2.35 | 1.93 | 1.94 | 2.60 | 3.27 |
| chr19 | 11.51 | 14.44 | 15.92 | 12.81 | 12.48 | 14.67 | 11.96 |
| chr20 | 3.70 | 4.73 | 4.90 | 3.92 | 4.75 | 6.06 | 5.39 |
| chr21 | 2.00 | 2.65 | 2.96 | 2.47 | 2.44 | 3.16 | 3.35 |
| chr22 | 4.84 | 6.12 | 6.60 | 5.08 | 5.28 | 6.00 | 4.86 |
| chrX | 1.15 | 2.85 | 1.57 | 1.28 | 1.49 | 2.21 | 4.01 |
| chrY | 0.38 | 0.11 | 0.48 | 0.45 | 0.53 | 0.44 | 0.12 |
| total | 2.92 | 4.05 | 4.18 | 3.39 | 3.79 | 4.28 | 4.45 |

Results of the Variant Calling Process is shown in Tables 16-19.

TABLE 16
Number of WES normal (PMC) germline variants

| Regions | metric | AAAAA | BBBBB | CCCCC | DDDDD | EVEEE | FFFFF | GGGGG |
|---|---|---|---|---|---|---|---|---|
| all | | | | | | | | |
| | number | 30,973 | 25,934 | 32,148 | 50,833 | 29,681 | 47,556 | 51,418 |
| | of | | | | | | | |
| | indels | | | | | | | |
| | number | 209,535 | 180,749 | 219,602 | 307,090 | 204,141 | 294,761 | 307,494 |
| | of | | | | | | | |
| | records | | | | | | | |
| | number | 178,629 | 154,862 | 187,522 | 256,349 | 174,505 | 247,278 | 256,162 |
| | of | | | | | | | |
| | SNPs | | | | | | | |
| CDS | | | | | | | | |
| | number | 1,105 | 1,061 | 1,104 | 1,120 | 1,055 | 1,110 | 1,066 |
| | of | | | | | | | |
| | indels | | | | | | | |
| | number | 33,753 | 33,422 | 33,595 | 33,764 | 33,364 | 33,733 | 33,402 |
| | of | | | | | | | |
| | records | | | | | | | |
| | number | 32,653 | 32,366 | 32,494 | 32,646 | 32,310 | 32,628 | 32,342 |
| | of | | | | | | | |
| | SNPs | | | | | | | |

TABLE 17
Number of WES tumor/normal somatic variants

| Regions | metric | AAAAA | BBBBB | CCCCC | DDDDD | EEEEE | FFFFF | GGGGG |
|---|---|---|---|---|---|---|---|---|
| all | | | | | | | | |
| | number of | 23 | 8 | 14 | 11 | 33 | 49 | 21 |
| | indels | | | | | | | |
| | number of | 288 | 163 | 605 | 619 | 305 | 3,329 | 180 |
| | records | | | | | | | |
| | number of | 265 | 155 | 591 | 608 | 272 | 3,280 | 159 |
| | SNPs | | | | | | | |
| CDS | | | | | | | | |
| | number of | 11 | 3 | 6 | 1 | 17 | 17 | 3 |
| | indels | | | | | | | |
| | number of | 204 | 85 | 344 | 346 | 132 | 1,528 | 66 |
| | records | | | | | | | |
| | number of | 193 | 82 | 338 | 345 | 115 | 1,511 | 63 |
| | SNPs | | | | | | | |

TABLE 18
Number of WGS normal germline variants

| Regions | metric | DDDDD | FFFFF | GGGGG |
|---|---|---|---|---|
| all | | | | |
| | number of indels | 931,451 | 934,986 | 939,511 |
| | number of records | 4,693,517 | 4,705,612 | 4,727,192 |
| | number of SNPs | 3,767,056 | 3,775,609 | 3,792,797 |
| CDS | | | | |
| | number of indels | 1,190 | 1,149 | 1,119 |
| | number of records | 34,546 | 34,431 | 34,649 |
| | number of SNPs | 33,359 | 33,287 | 33,536 |

TABLE 19
Number of WGS tumor/normal somatic variants

| Regions | metric | DDDDD | FFFFF | GGGGG |
|---|---|---|---|---|
| all | | | | |
| | number of indels | 3,186 | 7,884 | 928 |
| | number of records | 29,927 | 182,205 | 5,103 |
| | number of SNPs | 26,741 | 174,321 | 4,175 |
| CDS | | | | |
| | number of indels | 3 | 16 | 2 |
| | number of records | 366 | 1,570 | 54 |
| | number of SNPs | 363 | 1,554 | 52 |

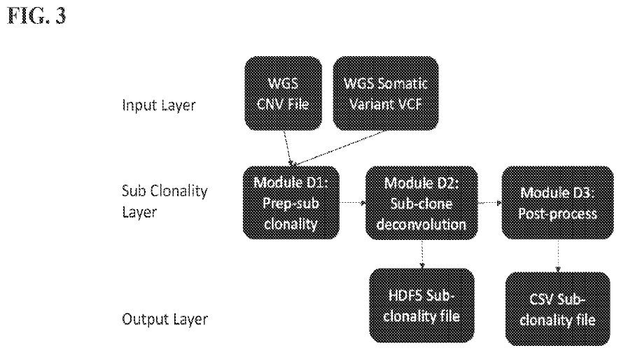

The subsequent module of workflow processed tumor somatic variants and copy number variants called by Module A from tumor-normal WGS samples and output a membership probability for each somatic variant into a set of N tumor-specific sub-clones, where N is a parameter output by the module. It also output an estimate of cellular prevalence for each mutation. This Module also performed bulk deconvolution of variants into sub-clones. The WGS files were generated from DRAGEN in Module A and processed in this module to estimate sub-clonality from cellular prevalence of somatic variants. FIG. 3 shows a workflow diagram of the module for clonality deconvolution.

1.3. Peptide Selection from NGS Data

Vaccine peptides to be manufactured were selected by a peptide prediction and machine learning algorithms within a personalize peptide prediction pipeline (p4vax). All components of this software solution are briefly described herein.

Mapping/Alignment, RNA gene expression, germline variants, somatic variants, and CNV caller output files, as well as the results of HLA haplotyping were ingested by the peptide prediction workflow to:

1. Identify variants with coding effects on expressed RNA

2. Select putative MHC class I and II binding peptides spanning across the non-synonymous variant

3. Confirm presence of variant in RNA sequencing reads

4. Estimate MHC class I and II processing, presentation, and immunogenicity

5. Confirm presence of mutation only in tumor DNA but not in germline (normal) DNA by correcting hg19 reference with germline variant calls

6. Filter potentially toxic peptides or products of endo- or exo-peptidase metabolism

7. Rank peptides on MHC class I and II processing, presentation, and immunogenicity

8. Maximize the expected percent of tumor cells targeted by the peptides

Specifically, for peptide selection, the results from WES and WES based somatic variant calling were used.

Using a tumor sub-clone deconvolution algorithm, the workflow processed tumor somatic variants & copy number variants called by Module A from tumor-normal WGS samples and additionally output a membership probability for each somatic variant into a set of N tumor-specific clusters of mutation/cellular prevalence. The results from this step were used in step 8.

The output of the peptide prediction pipeline is an exhaustive list of potential vaccine peptides ranked by a combination of MHC class I and II binding/presentation scores, immunogenicity, and tumor cellular prevalence clusters.

From this ranked list of potential peptide synthesis candidates, a set of up to 80 peptides ranked highest in the list was selected. After manual verification of the existence of somatic variants, peptide sequences resulting from verified somatic variants were communicated to a peptide manufacturer for synthesis.

Peptide Selection was performed for the Work Example samples DDDDD, FFFFF, and GGGGG (Table 20-Table 22) and twenty peptides were selected further as candidates for pool formulation.

TABLE 20
Peptide Selection for engineering run sample DDDDD

| | | | | | | SEQ | | |
|---|---|---|---|---|---|---|---|---|
| Short/ | | | | | | ID | MHC I | MHC II |
| Lon | Rank | Gene | Variant | Sequence | Length | NO: | score | score |
| long | 1 | MOGS | chr2: 74690507: C: T | QDSNTSALPLVSLFF | 15 | 2 | 0.07329569 | 0.66997391 |
| | 2 | EXOC7 | chr17: 74079782: C: T | YIKYKVEQVGDMIDRL | 16 | 3 | 0.05583751 | 0.62314493 |
| | 3 | MEGF8 | chr19: 42858849: C: T | GQRRDRLLTVQALSGL | 16 | 4 | 0.05151247 | 0.73578168 |
| | 4 | EXOC7 | chr17: 74079781: C: T | YIKYKVEQVGDMIDRLF | 17 | 5 | 0.05031836 | 0.63131081 |
| | 5 | LIMA1 | chr12: 50571454: G: A | EGIKMSKPKWPLEDEISKPE | 22 | 6 | 0.20668915 | 0.43862386 |
| | | | | VP | | | | |
| | 6 | PTPN4 | chr2: 120714433: A: C | QFDQLYRTKPGMTMSC | 16 | 7 | 0.03901816 | 0.65768236 |
| | 7 | BRAF | chr7: 140414433: A: C | GDVAVKMLTPQQLQA | 15 | 8 | 0.0365321 | 0.67463777 |
| | | | TAGGTGCTGTCACAT | | | | | |
| | | | (SEQ ID NO: 1): G | | | | | |
| | 8 | GINS4 | chr8: 41393899: T: A | RLEQAWMNEKLAPELLESKP | 20 | 9 | 0.03424379 | 0.51320203 |
| | 9 | GAS8 | chr16: 90106895: C: G | NEVLAASNLDPAALRLVSRK | 22 | 10 | 0.02816643 | 0.55796491 |
| | | | | LE | | | | |
| | 10 | PRKCZ | chr1: 2066754: G: A | GARRWRKLYRANSHLFQAKR | 22 | 11 | 0.0278652 | 0.57790382 |
| | | | | FN | | | | |
| | 11 | RPS6KB2 | chr11: 67200282: C: T | SQGIIYRDLKLENIMLSSQG | 22 | 12 | 0.13305076 | 0.58418771 |
| | | | | HI | | | | |
| | 12 | BST1 | chr4: 15716974: C: T | GSEPTGAYSIKGFFADYEIP | 21 | 13 | 0.02372363 | 0.73712491 |
| | | | | N | | | | |
| | 13 | HIPK2 | chr7: 139316445: G: A | YRAPEIILGLLFCEAI | 16 | 14 | 0.02345472 | 0.78064901 |
| | 14 | SYNJ1 | chr21: 34017988: G: A | APPTRLAPPQRPPPPS | 16 | 15 | 0.01945223 | 0.46290062 |
| | 15 | EPS8L3 | chr1: 110293322: C: T | LQMLCPQEAPQILSRLEAVR | 21 | 16 | 0.09542136 | 0.6229403 |
| | | | | R | | | | |
| | 16 | TRIM13 | chr13: 5086534: G: A | SLFQSFETWHRGDALSRL | 18 | 17 | 0.01839422 | 0.6919829 |
| | 17 | CPNE3 | chr8: 875541180: G: A | SGQQWYEVEHTERIKNCLN | 19 | 18 | 0.08889867 | 0.46731711 |
| | 18 | CPQ | chr8: 98155388: G: A | AVVSYVVADMKEMLPRS | 17 | 19 | 0.01565236 | 0.68498519 |
| | 19 | BDP1 | chr5: 70766293: C: T | GQLFPYRARIEKIKNKF | 16 | 20 | 0.01549436 | 0.71614594 |
| | 20 | ANF784 | chr19: 56135881: G: A | ARPEAQSRSSPTLES | 15 | 21 | 0.01368863 | 0.43412852 |
| | 21 | FOS | chr14: 75748046: T: G | TYPEADSFPSWAAAHRKGS | 19 | 22 | 0.06817705 | 0.59480153 |
| | 22 | FAMI34A | chr2: 220047223: C: A | FELLDQGELEKLNAELGLEP | 20 | 23 | 0.06638302 | 0.50878612 |
| | 23 | LTK | chr15: 41796285: G: A | EKLKSWGGSLLGPWLSSGLK | 22 | 24 | 0.01248521 | 0.57080845 |
| | | | | PL | | | | |
| | 24 | SMC2 | chr9: 106885498: C: T | YHKQQEELDAFKKTIEESEE | 22 | 25 | 0.01245417 | 0.52025989 |
| | | | | TL | | | | |
| | 25 | UAP1L1 | chr9: 139976457: C: T | HGAWLPELPSLPSNGDPPAI | 22 | 26 | 0.01167158 | 0.48069739 |
| | | | | CE | | | | |
| | 26 | ASCC3 | chr6: 101296376: A: C | QDDFTALGQMTEKEHG | 16 | 27 | 0.01114204 | 0.54359088 |
| | 27 | DMAP1 | chr1: 44679478: G: A | RDILELGGPEEDAASGTIS | 19 | 28 | 0.01011638 | 0.5441813 |
| | 28 | NUP98 | chr11: 3765801: C: T | GPLGTGAFEAPGFNTTTATL | 22 | 29 | 0.00959293 | 0.67302464 |
| | | | | GF | | | | |
| | 29 | SPOCK1 | chr5: 136328211: C: T | FGALHEDANKVIKPTSSNTA | 21 | 30 | 0.00848382 | 0.4601863 |
| | | | | Q | | | | |
| | 30 | EEF1A1 | chr6: 74227798: C: T | KSGDAAIVDMVPSKPMCVES | 22 | 31 | 0.04120537 | 0.605315 |
| | | | | FS | | | | |
| | 31 | ANF697 | chr1: 120166583: C: G | REDDDESAGENPLEEEEEQP | 22 | 32 | 0.00771731 | 0.41146906 |
| | | | | AP | | | | |
| | 32 | MOGS | chr2: 74690506: C: T | SWRVTVEPQDSNTSALPLVS | 22 | 33 | 0.00759169 | 0.55339174 |
| | | | | LF | | | | |
| | 33 | CIC | chr19: 42796535: C: T | PASSQAGTVTLYGPTSSVAL | 20 | 34 | 0.00728918 | 0.62779866 |
| | 34 | MEPCE | chr7: 100029289: C: T | DGADTSVFSNNVVFVTGNYV | 22 | 35 | 0.00681793 | 0.70873332 |
| | | | | LD | | | | |
| | 35 | MDN1 | chr6: 90359807: C: T | WLRRTKPSKHQYQICLAIDD | 22 | 36 | 0.00677077 | 0.61246852 |
| | | | | SS | | | | |
| | 36 | RBM42 | chr19: 36120481: T: G | VPGIPTAVPAGPTVPTVPTV | 22 | 37 | 0.03277055 | 0.4079915 |
| | | | | EA | | | | |
| | 37 | MMKI67 | chr10: 129901135: G: A | PVKSQSKSNTFLPPLPFKRG | 20 | 38 | 0.0057615 | 0.57937322 |
| | 38 | TEAD3 | chr6: 35454323: G: A | GWTTMRRACGARTLSRA | 17 | 39 | 0.00532379 | 0.68094877 |
| | 39 | FNBP1L | chr1: 94012476: T: C | MGDPGSLQPKSAETMNNIDR | 22 | 40 | 0.00510709 | 0.44982782 |
| | | | | LR | | | | |
| | 40 | EXTL3 | chr8: 28574907: C: T | DPRLVIFSGCATRLFE | 16 | 41 | 0.00393086 | 0.81055314 |
| | 41 | EIF3F | chr11: 8013351: G: A | KHSVKVTNCFSVPHN | 15 | 42 | 0.0096035 | 0.66676886 |
| | 42 | ATP13A1 | chr19: 19762504: C: T | EKAHTLILQPPSKKGRQCEW | 22 | 43 | 0.00842387 | 0.49265976 |
| | | | | RS | | | | |
| short | 1 | EXOC7 | chr17: 74079781: C: T | YIKYKVEQV | 9 | 44 | 0.04508725 | N/A |
| | 2 | LIMA1 | chr12: 50571454: G: A | LEDEISKPEV | 10 | 45 | 0.16698617 | N/A |
| | 3 | PRKCZ | chr1: 2066754: G: A | YRANSHLF | 8 | 46 | 0.01988399 | N/A |
| | 4 | BST1 | chr4: 15716974: C: T | SIKGFFADY | 9 | 47 | 0.01757893 | N/A |
| | 5 | MEGF8 | chr19: 42858849: C: T | QRRDRLLTV | 9 | 48 | 0.02304197 | N/A |
| | 6 | CPQ | chr8: 98155388: G: A | YVVADMKEM | 9 | 49 | 0.01129116 | N/A |

TABLE 21
Peptide selection for engineering run sample FFFFF

| | | | | | SEQ | | | |
|---|---|---|---|---|---|---|---|---|
| Short/ | | | | | ID | | MHC I | MHC II |
| Lon | Rank | Gene | Variant | Sequence | NO: | Length | score | score |
| long | 1 | SLC45A4 | chr8: 142264105: G: A | AMETALVTLILLQI | 50 | 17 | 0.16258827 | 0.73463601 |
| | | | | GKS | | | | |
| | 2 | PWP2 | chr21: 45534552: C: T | KGNIAQMYHAFGKK | 51 | 22 | 0.14479999 | 0.68694234 |
| | | | | REFNAFVL | | | | |
| | 3 | PGAP1 | chr2: 197744849: G: A | KYLTLRLQDYLSLS | 52 | 20 | 0.13617044 | 0.81953448 |
| | | | | HLVVYV | | | | |
| | 4 | MFSD2A | chr1: 40424437: C: A | GRAWDAITDHLVGL | 53 | 20 | 0.13150237 | 0.6182176 |
| | | | | CISKSP | | | | |
| | 5 | SLC39A7 | chr6: 33171427: C: T | VGSEIAGGAGLGWV | 54 | 22 | 0.62906294 | 0.64794417 |
| | | | | LPFTAGGF | | | | |
| | 6 | ZCCHC4 | chr4: 25335077: C: T | GFRRVLCVGTLRLH | 55 | 19 | 0.11540349 | 0.84718676 |
| | | | | ELIKL | | | | |
| | 7 | THADA | chr2: 43802077: G: A | SAIQVLESSSLSLT | 56 | 18 | 0.11353184 | 0.75960565 |
| | | | | DSLN | | | | |
| | 8 | INO80D | chr2: 206921080: G: A | LPLLLFSRAPTVDP | 57 | 16 | 0.11332213 | 0.7355256 |
| | | | | PR | | | | |
| | 9 | INO80D | chr2: 206921083: G: A | LPLLLFSRAPTVDP | 58 | 15 | 0.11291208 | 0.73622152 |
| | | | | P | | | | |
| | 10 | FAM208B | chr10: 5790009: C: T | AKCTGDFSPSLEKL | 59 | 21 | 0.11199412 | 0.64350711 |
| | | | | VKSGNPL | | | | |
| | 11 | PCM1 | chr8: 17813087: C: T | GESNSLTSSVLYPT | 60 | 22 | 0.55904726 | 0.80863367 |
| | | | | ASLVSQNE | | | | |
| | 12 | COL18A1 | chr21: 46910199: G: A | GAKGEVEADGIPGF | 61 | 18 | 0.52507762 | 0.57400138 |
| | | | | PGLP | | | | |
| | 13 | NUFIP2 | chr17: 27613951: G: A | PVDNSSAKIVLKIS | 62 | 21 | 0.48588871 | 0.69306097 |
| | | | | YASKVKE | | | | |
| | 14 | INTS1 | chr7: 1517425: G: A | VVVSSLLLQEEELL | 63 | 22 | 0.46108 | 0.62556372 |
| | | | | AGGKPGAD | | | | |
| | 15 | NDUFB10 | chr16: 2011845: CAG: | ARKCLAKQAEDAAR | 64 | 21 | 0.46096059 | 0.49336677 |
| | | | C | EKSCKRG | | | | |
| | 16 | BCAP29 | chr7: 107234541: T: G | RNLYISGFSLCFWL | 65 | 22 | 0.41204663 | 0.78909355 |
| | | | | VLRRLVTL | | | | |
| | 17 | CXorf40B | chrX: 149100859: G: C | KQKYLTVISNRRWL | 66 | 21 | 0.32483088 | 0.74806946 |
| | | | | LEPIPRK | | | | |
| | 18 | SCARB1 | chr12: 125284765: G: | RFSAPLFLSHSHFL | 67 | 21 | 0.31241171 | 0.74533032 |
| | | | A | NADPVLA | | | | |
| | 19 | PTPRZ1 | chr7: 121694076: C: T | WDHNAQLVVMILDG | 68 | 22 | 0.30682347 | 0.65887653 |
| | | | | QNMAEDEF | | | | |
| | 20 | TBC1D23 | chr3: 100014007: A: G | AIWDGYLQQAGPFF | 69 | 22 | 0.29311405 | 0.58318637 |
| | | | | IYFLMLII | | | | |
| | 21 | SYNE1 | chr6: 152638090: A: C | ASHLEEYNERLELI | 70 | 18 | 0.28917584 | 0.56130198 |
| | | | | LKWI | | | | |
| | 22 | MBTPS1 | chr16: 84115485: G: A | KRELVNSASMKQAL | 71 | 21 | 0.27998486 | 0.87108388 |
| | | | | IASARRL | | | | |
| | 23 | POLR2B | chr4: 57861015: AT: A | PLDPGGYFIIMDQK | 72 | 16 | 0.27682839 | 0.82729797 |
| | | | | RF | | | | |
| | 24 | GOLGB1 | chr3: 121415783: A: G | LQEALTSRKAIPKK | 73 | 22 | 0.27224502 | 0.48823824 |
| | | | | AQEKERHL | | | | |
| | 25 | PPP4R2 | chr3: 73112870: C: T | LNRMNGVMFSGNSP | 74 | 22 | 0.23383287 | 0.77749593 |
| | | | | SYTERSNI | | | | |
| | 26 | FARP2 | chr2: 242403325: C: T | QRLALWEGPFKAHT | 75 | 19 | 0.22494294 | 0.7763395 |
| | | | | KGSHQ | | | | |
| | 27 | MRPL38 | chr17: 73898232: G: A | REYFGEKTKLKEKI | 76 | 21 | 0.21683746 | 0.663767 |
| | | | | DIGKPPP | | | | |
| | 28 | FLYWCH1 | chr16: 2980828: G: A | PALEEEEAPQALSL | 77 | 22 | 0.2157846 | 0.73017404 |
| | | | | LSLPPKKR | | | | |
| | 29 | PHF12 | chr17: 27244419: G: A | LSNRCQVFDCFQDT | 78 | 20 | 0.21377716 | 0.68927787 |
| | | | | VSQHVV | | | | |
| | 30 | ITGA7 | chr12: 56096837: C: T | DCYRVDIDQEADMQ | 79 | 19 | 0.16359662 | 0.5337592 |
| | | | | KESKE | | | | |
| | 31 | DDX2 | chr20: 47835942: G: A | RRRGGCEKLRAEPQ | 80 | 21 | 0.15278271 | 0.78941247 |
| | | | | AVLASGS | | | | |
| | 32 | RBL2 | chr16: 53499357: A: G | HFYKVIGVFIRAED | 81 | 17 | 0.14770824 | 0.83922687 |
| | | | | GLC | | | | |
| | 33 | NISCH | chr3: 52526301: C: T | ALTLVFDDVQGYDL | 82 | 20 | 0.14695032 | 0.72112204 |
| | | | | MGSVTL | | | | |
| | 34 | CDK5RAP3 | chr17: 46058540: G: A | MILASPRYVDQVTE | 83 | 22 | 0.1403607 | 0.74608702 |
| | | | | FLQQKLKQ | | | | |
| | 35 | TANK | chr2: 162061206: C: T | NSTQDNNYGCVSLL | 84 | 22 | 0.14031428 | 0.62717317 |
| | | | | EDSETRKN | | | | |
| | 36 | SPTBN1 | chr2: 54886355: C: T | EEEERKRRPPSLEP | 85 | 22 | 0.13262047 | 0.55289858 |
| | | | | STKVSEEA | | | | |
| | 37 | VASP | chr19: 46025750: C: T | AGGGPPPAPPLSAA | 86 | 22 | 0.13190385 | 0.53188621 |
| | | | | QGPGGGGA | | | | |
| | 38 | PRPF | chr6: 4052261: C: T | KLNDADPDDKFYCL | 87 | 22 | 0.12311099 | 0.63227453 |
| | | | | RLFRHFYH | | | | |
| | 39 | TNPO1 | chr5: 72147095: C: T | VQQKLEQLNQYSDF | 88 | 22 | 0.11718017 | 0.63499542 |
| | | | | NNYLIFVL | | | | |
| | 40 | USP11 | chrX: 47104877: C: T | TTVETLEKENSWYC | 89 | 20 | 0.11154779 | 0.61009519 |
| | | | | PSCKQH | | | | |
| | 41 | RPS24 | chr10: 79814617: T: G | VDGDWVLHLPEALS | 90 | 15 | 0.10728163 | 0.90107272 |
| | | | | A | | | | |
| | 42 | PRRC2B | chr134351481: C: T | RFRRLRQEREFLGL | 91 | 18 | 0.08403133 | 0.71237872 |
| | | | | WGPE | | | | |
| short | 1 | SLC45A4 | chr8: 142264105: G: A | METALVTLIL | 92 | 10 | 0.08417078 | N/A |
| | 2 | PCM1 | chr8: 17813087: C: T | SVLYPTASL | 93 | 9 | 0.29510115 | N/A |
| | 3 | SLC45A4 | chr8: 142264105: G: A | ALVTLILLQI | 94 | 10 | 0.07058771 | N/A |
| | 4 | COL18A1 | chr21: 46910199: G: A | EADGIPGFPGL | 95 | 11 | 0.28469103 | N/A |
| | 5 | COL18A1 | chr21: 46910199: G: A | GEVEADGIPGF | 96 | 11 | 0.30583803 | N/A |
| | 6 | TBC1D9 | chr4: 141545323: G: A | RLWTSENKSK | 97 | 10 | 0.04554415 | N/A |

TABLE 22
Peptide Selection for Engineering Run Sample GGGGG

| | | | | | | SEQ | | |
|---|---|---|---|---|---|---|---|---|
| Short/ | | | | | | ID | MCH I | MHC II |
| Lon | Rank | Gene | Variant | Sequence | Length | NO. | score | score |
| long | 1 | TSN | chr2: 122519017: G: A | HEHWRFVLQHLVFL | 19 | 98 | 0.0464269 | 0.76456928 |
| | | | | AAFVV | | | | |
| | 2 | MED9 | chr17: 17380542: T: C | QSPARAREEENHSF | 22 | 99 | 0.02823036 | 0.84198684 |
| | | | | LPLVHNII | | | | |
| | 3 | RBM28 | chr7: 127975934: G: A | EDEEEENIELKVTK | 17 | 100 | 0.12394809 | 0.50573551 |
| | | | | PVQ | | | | |
| | 4 | PRKDC | chr8: 48690396: G: A | PELMPFRFLTCQFI | 21 | 101 | 0.07648852 | 0.76358768 |
| | | | | NLMLPMKE | | | | |
| | 5 | ZNF12 | chr7: 6737368: C: G | EEWQQLDPEQNITY | 22 | 102 | 0.01395768 | 0.54926563 |
| | | | | RDVMLENY | | | | |
| | 6 | MRPL48 | chr11: 73575383: G: A | DFKGRFKARPKLEE | 20 | 103 | 0.01373842 | 0.6212347 |
| | | | | LLAKLK | | | | |
| | 7 | INTS1 | chr7: 1526709: G: A | ASSQSMPWLVDLVQ | 22 | 104 | 0.05733778 | 0.62354692 |
| | | | | SSEGSLDV | | | | |
| | 8 | DNAJC10 | chr2: 183622543: A: G | RFFPPKSNKACHYH | 21 | 105 | 0.05379868 | 0.63486407 |
| | | | | SYNGWNR | | | | |
| | 9 | MEN1 | chr11: 64573759: G: A | MYLAGYHCRNCNVR | 21 | 106 | 0.00744509 | 0.8061702 |
| | | | | EALQAWA | | | | |
| | 10 | SNX25 | chr4: 186185720: C: T | LVFCLLPSKDVQFL | 15 | 107 | 0.00702811 | 0.83040282 |
| | | | | S | | | | |
| | 11 | LCMT1 | chr16: 25180500: C: T | RRRQCDLVGVETCK | 19 | 108 | 0.00388609 | 0.65396226 |
| | | | | SLESQ | | | | |
| | 12 | INF2 | chr14: 105179557: G: | FLRALKENKDQKEQ | 21 | 109 | 0.0161754 | 0.43941237 |
| | | | A | AAKAERR | | | | |
| short | 1 | RSN | chr2: 122519017: G: A | FVLQHLVFL | 9 | 110 | 0.03300495 | N/A |
| | 2 | MED9 | chr17: 17380542: T: C | REEENHSFL | 9 | 111 | 0.01610038 | N/A |
| | 3 | MED9 | chr17: 17380542: T: C | REEENHSFLPL | 11 | 112 | 0.01619894 | N/A |
| | 4 | TSN | chr2: 122519017: G: A | VLQHLVFLA | 9 | 113 | 0.01469447 | N/A |
| | 5 | MED9 | chr17: 17380542: T: C | EENHSFLPL | 9 | 114 | 0.00951051 | N/A |
| | 6 | INTS1 | chr7: 1526709: G: A | SMPWLVDLV | 9 | 115 | 0.04252224 | N/A |
| | 7 | MED9 | chr17: 17380542: T: C | RAREEENHSF | 10 | 116 | 0.0088703 | N/A |
| | 8 | MRPL48 | chr11: 73575383: G: A | KARPKLEEL | 9 | 117 | 0.00901679 | N/A |
| | 9 | TSN | chr2: 122519017: G: A | FVLQHLVFLA | 10 | 118 | 0.00723975 | N/A |
| | 10 | PRKDC | chr8: 48690396: G: A | RLTCQFINL | 9 | 119 | 0.03565368 | N/A |
| | 11 | ZNF12 | chr7: 6737368: C: G | QLDPEQNITY | 10 | 120 | 0.0119429 | N/A |
| | 12 | RBM28 | chr7: 127975934: G: A | NIELKVTKPVQ | 11 | 121 | 0.05185511 | N/A |
| | 13 | MED9 | chr17: 17380542: T: C | REEENHSF | 8 | 122 | 0.00539441 | N/A |
| | 14 | MED9 | chr17: 17380542: T: C | EEENHSFLPL | 10 | 123 | 0.00433009 | N/A |
| | 15 | RBM28 | chr7: 127975934: G: A | NIELKVTKPV | 10 | 124 | 0.03534479 | N/A |
| | 16 | TSN | chr2: 122519017: G: A | FVLQHLVF | 8 | 125 | 0.0063978 | N/A |
| | 17 | RBM28 | chr7: 127975934: G: A | DEEEENIELKV | 11 | 126 | 0.04180784 | N/A |
| | 18 | RBM28 | chr7: 127975934: G: A | EEEENIELKV | 10 | 127 | 0.03181995 | N/A |
| | 19 | MED9 | chr17: 17380542: T: C | RAREEENHSFL | 11 | 128 | 0.00303641 | N/A |
| | 20 | TSN | chr2: 122519017: G: A | VLQHLVFLAA | 10 | 129 | 0.0029588 | N/A |
| | 21 | RBM28 | chr7: 127975934: G: A | EEEENIELKVT | 11 | 130 | 0.02003145 | N/A |
| | 22 | INTS1 | chr7: 1526709: G: A | QSMPWLVDLV | 10 | 131 | 0.01398635 | N/A |
| | 23 | PRKDC | chr8: 48690396: G: A | LTCQFINLM | 9 | 132 | 0.01486982 | N/A |
| | 24 | RBM28 | chr7: 127975934: G: A | EEENIELKVTK | 11 | 133 | 0.01899035 | N/A |
| | 25 | TSN | chr2: 122519017: G: A | VLQHLVFL | 8 | 134 | 0.00307205 | N/A |
| | 26 | SNX25 | chr4: 186185720: C: T | LLPSKDVQFL | 10 | 135 | 0.00218817 | N/A |
| | 27 | RBM28 | chr7: 127975934: G: A | EDEEEENIELK | 11 | 136 | 0.00853576 | N/A |
| | 28 | ZNF12 | chr7: 6737368: C: G | QLDPEQNITYR | 11 | 137 | 0.00213218 | N/A |
| | 29 | INF2 | chr14: 105179557: G: | ALEKNKDQK | 9 | 138 | 0.00667427 | N/A |
| | | | A | | | | | |
| | 30 | MRPL48 | chr11: 73575383: G: A | KLEELLAK | 8 | 139 | 0.0020238 | N/A |
| | 31 | MED9 | chr17: 17380542: T: C | AREEENHSFLP | 11 | 140 | 0.00150676 | N/A |
| | 32 | DNAJC10 | chr2: 183622543: A: G | KSNKACHY | 8 | 141 | 0.01944355 | N/A |
| | 33 | RBM28 | chr7: 127975934: G: A | PSKDVQFL | 8 | 142 | 0.00137019 | N/A |
| | 34 | PADRE | N/A | EEEENIELK | 9 | 143 | 0.00556938 | N/A |
| | | PADRE | N/A | AKFVAAWTLKAAA | 13 | 144 | N/A | N/A |
| | | | | AKFVAAWTLKAAA | 13 | 145 | N/A | N/A |

### 1.4. Peptide Manufacturing

Vaccine peptide manufacturing and pool formulation occured at a peptide manufacturer performing peptide synthesis, quality control, dissolving and mixing of peptides and peptide pools.

Peptides were prepared by solid phase peptide synthesis, purified using RP-HPLC columns, and analyzed for quality (identity, purity, peptide content, Acetate/TFA content, residual organic solvents).

Results of the peptide synthesis are shown in Tables 23 to 25.

TABLE 23
Peptide Synthesis Engineering Run Batch for Sample DDDDD_2020

| | Peptide | Peptide | | SEQ | | Peptide | Final | Passed | |
|---|---|---|---|---|---|---|---|---|---|
| No. | ID | Lot | Peptide Sequence | ID NO: | Length | Classss | Lyo | QC | Solubility |
| 1 | DDDDD.1 | DDDDD.1 | PASSQAGTVTLYGPTSSVALGF | 146 | 25 | Class | N/A | N/A | N/A |
| | | | TSL | | | I/II | | | |
| 2 | DDDDD.2 | DDDDD.2 | EDDDESAGENPLEEEEEQPAP | 147 | 21 | Class | N/A | N/A | N/A |
| | | | | | | I/II | | | |
| 3 | DDDDD.3 | DDDDD.2 | MSKPKWPLEDEISKPEVP | 148 | 18 | Class | Y | Y | Clear |
| | | | | | | I/II | | | |
| 4 | DDDDD.4 | DDDDD.4 | NQPKIGGPLGTGAFEAPGFNTT | 149 | 28 | Class | Y | Y | Clear |
| | | | TATLGF | | | I/II | | | |
| 5 | DDDDD.5 | DDDDD.5 | FGALHEDANKVIKPTSSNTA | 150 | 20 | Class | Y | Y | Clear |
| | | | | | | I/II | | | |
| 6 | DDDDD.6 | DDDDD.6 | KNPQMGDPGSLQPKSAETMNN | 151 | 28 | Class | Y | Y | Clear |
| | | | IDRLRME | | | I/II | | | |
| 7 | DDDDD.7 | DDDDD.7 | LQMLCPQEAPQILSRLEAVR | 152 | 20 | Class | Y | Y | Clear |
| | | | | | | I/II | | | |
| 8 | DDDDD.8 | DDDDD.8 | YVMVVMSDSSIPSAATLINIRN | 153 | 23 | Class | N/A | N/A | N/A |
| | | | A | | | I/II | | | |
| 9 | DDDDD.9 | DDDDD.9 | SSCMGGMNQRPILTIITLED | 154 | 20 | Class | Y | Y | Cloudy |
| | | | | | | I/II | | | |
| 10 | DDDDD.10 | DDDDD.10 | YRAPEIILGLLFCEAI | 155 | 16 | Class | N/A | N/A | N/A |
| | | | | | | I/II | | | |
| 11 | DDDDD.11 | DDDDD.11 | DPRLVIFSGCATRLFEA | 156 | 17 | Class | Y | Y | Cloudy |
| | | | | | | I/II | | | |
| 12 | DDDDD.12 | DDDDD.12 | ARPEAQSRSSPTLES | 157 | 15 | Class | Y | Y | Clear |
| | | | | | | I/II | | | |
| 13 | DDDDD.13 | DDDDD.13 | DGADTSVFSNNVVFVT | 158 | 16 | Class | N/A | N/A | N/A |
| | | | | | | I/II | | | |
| 14 | DDDDD.14 | DDDDD.14 | GQRRDRLLTVQALSGL | 159 | 16 | Class | Y | Y | Clear |
| | | | | | | I/II | | | |
| 15 | DDDDD.15 | DDDDD.15 | WLPELPSLPSNGDPPAICE | 160 | 19 | Class | Y | Y | Clear |
| | | | | | | I/II | | | |
| 16 | DDDDD.16 | DDDDD.16 | LLGTVDKHSVKVTNCFSVPHN | 161 | 21 | Class | Y | Y | Clear |
| | | | | | | I/II | | | |
| 17 | DDDDD.17 | DDDDD.17 | KDKIWLRRTKPSKHQYQICLAI | 162 | 28 | Class | Y | Y | Clear |
| | | | DDSSSM | | | I/II | | | |
| 18 | DDDDD.18 | DDDDD.18 | QELSPEKLKSWGGSLLGPWLSS | 163 | 28 | Class | Y | Y | Clear |
| | | | GLKPLK | | | I/II | | | |
| 19 | DDDDD.19 | DDDDD.19 | ESADLPPKGFQASYGKDE | 164 | 18 | Class | N/A | N/A | N/A |
| | | | | | | I/II | | | |
| 20 | DDDDD.20 | DDDDD.20 | TYPEADSFPSWAAAHRKGSS | 165 | 20 | Class | Y | Y | Clear |
| | | | | | | I/II | | | |
| 21 | DDDDD.21 | DDDDD.21 | RDAFESLFQSFETWHRGDALSR | 166 | 24 | Class | Y | Y | Semi- |
| | | | LD | | | I/II | | | cloudy |
| 22 | DDDDD.22 | DDDDD.22 | APPTRLAPPQRPPPPS | 167 | 16 | Class | Y | Y | Clear |
| | | | | | | I/II | | | |
| 23 | DDDDD.23 | DDDDD.23 | MWSSINCIICACVKGR | 168 | 16 | Class | N/A | N/A | N/A |
| | | | | | | I/II | | | |
| 24 | DDDDD.24 | DDDDD.24 | RRWRKLYRANSHLFQAKRFN | 169 | 20 | Class | Y | Y | Clear |
| | | | | | | I/II | | | |
| 25 | DDDDD.25 | DDDDD.25 | AVVSYVVADMKEMLPRS | 170 | 17 | Class | Y | Y | Clear |
| | | | | | | I/II | | | |
| 26 | DDDDD.26 | DDDDD.26 | LNGSEPTGAYSIKGFFADYEIP | 171 | 23 | Class | Y | Y | Clear |
| | | | N | | | I/II | | | |
| 27 | DDDDD.27 | DDDDD.27 | GQLFPYRARIEIKNKF | 172 | 16 | Class | Y | Y | Clear |
| | | | | | | I/II | | | |
| 28 | DDDDD.28 | DDDDD.28 | QFDQLYRTKPGMTMSC | 173 | 16 | Class | N/A | N/A | N/A |
| | | | | | | I/II | | | |
| 29 | DDDDD.29 | DDDDD.29 | PRTESSDVADQLWAQ | 174 | 15 | Class | Y | Y | Semi- |
| | | | | | | I/II | | | cloudy |
| 30 | DDDDD.30 | DDDDD.30 | RDILELGGPEEDAASGTISKKD | 175 | 22 | Class | Y | Y | Clear |
| | | | | | | I/II | | | |
| 31 | DDDDD.31 | DDDDD.31 | SQAGTVTL | 176 | 8 | Class | Y | Y | Clear |
| | | | | | | I | | | |
| 32 | DDDDD.32 | DDDDD.32 | QSRSSPTL | 177 | 8 | Class | N/A | N/A | N/A |
| | | | | | | I | | | |
| 33 | DDDDD.33 | DDDDD.33 | KVTNCFSV | 178 | 8 | Class | Y | Y | Clear |
| | | | | | | I | | | |
| 34 | DDDDD.34 | DDDDD.34 | TWHRGDAL | 179 | 8 | Class | Y | Y | Clear |
| | | | | | | I | | | |
| 35 | DDDDD.35 | DDDDD.35 | SSQAGTVTL | 180 | 9 | Class | Y | Y | Semi- |
| | | | | | | I | | | cloudy |
| 36 | DDDDD.36 | DDDDD.36 | AQSRSSPTL | 181 | 9 | Class | Y | Y | Clear |
| | | | | | | I | | | |
| 37 | DDDDD.37 | DDDDD.37 | RRACGARTL | 182 | 9 | Class | Y | Y | Clear |
| | | | | | | I | | | |
| 38 | DDDDD.38 | DDDDD.38 | ASSQAGTVTL | 183 | 10 | Class | Y | Y | Clear |
| | | | | | | I | | | |
| 39 | DDDDD.39 | DDDDD.39 | RAPEIILGLL | 184 | 10 | Class | Y | Y | Semi- |
| | | | | | | I | | | cloudy |
| 40 | DDDDD.40 | DDDDD.40 | EAQSRSSPTL | 185 | 10 | Class | Y | Y | Clear |
| | | | | | | I | | | |
| 41 | DDDDD.41 | DDDDD.41 | NTSALPLVSL | 186 | 10 | Class | Y | Y | Clear |
| | | | | | | I | | | |
| 42 | DDDDD.42 | DDDDD.42 | DSSIPSAATL | 187 | 10 | Class | Y | Y | Clear |
| | | | | | | I | | | |
| 43 | DDDDD.43 | DDDDD.43 | SAGENPLEEEE | 188 | 11 | Class | Y | Y | Clear |
| | | | | | | I | | | |
| 44 | DDDDD.44 | DDDDD.44 | NSPSTPTEQRI | 189 | 11 | Class | N/A | N/A | N/A |
| | | | | | | I | | | |
| 45 | DDDDD.45 | DDDDD.45 | FEAPGFNTTTA | 190 | 11 | Class | Y | Y | Clear |
| | | | | | | I | | | |
| 46 | DDDDD.46 | DDDDD.46 | AKFVAAWTLKAAA | 191 | 13 | PADRE | Y | Y | Clear |
| 47 | DDDDD.47 | DDDDD. | AKFVAAWTLKAAA | 192 | 13 | PADRE | Y | Y | Clear |
| | | 46-1 | | | | | | | |
| 48 | DDDDD.48 | DDDDD. | AKFVAAWTLKAAA | 193 | 13 | PADRE | Y | Y | Clear |
| | | 46-2 | | | | | | | |

TABLE 24
Peptide synthesis engineering run batch for sample FFFFF

| | Peptide | Peptide | | SEQ | | Peptide | Final | Passed | |
|---|---|---|---|---|---|---|---|---|---|
| No. | ID | Lot | Peptide Sequence | ID NO: | Length | Class | Lyo | QC | Solubility |
| 1 | FFFFF.1 | FFFFF.1 | LFMLTFSTSPGLESPVESFIAFLLI | 194 | 25 | Class | N/A | N/A | N/A |
| | | | | | | I/II | | | |
| 2 | FFFFF.2 | FFFFF.2 | PVVNGESNSLTSSVLYPTASLVSQN | 195 | 25 | Class | N/A | N/A | N/A |
| | | | | | | I/II | | | |
| 3 | FFFFF.3 | FFFFF.3 | SARKCLAKQAEDAAREKSCK | 196 | 20 | Class | N/A | N/A | N/A |
| | | | | | | I/II | | | |
| 4 | FFFFF.4 | FFFFF.4 | TNSAIQVLESSSLSLTDSLNGNS | 197 | 23 | Class | N/A | N/A | N/A |
| | | | | | | I/II | | | |
| 5 | FFFFF.5 | FFFFF.5 | PTLPQPASHFSPPPPPPPLPP | 198 | 21 | Class | Y | Y | Clear |
| | | | | | | I/II | | | |
| 6 | FFFFF.6 | FFFFF.6 | FINPIFEFSQAMRRLGLDDA | 199 | 20 | Class | Y | N | N/A |
| | | | | | | I/II | | | |
| 7 | FFFFF.7 | FFFFF.7 | STVQKRELVNSASMKQALIASARRLP | 200 | 26 | Class | Y | N/A | N/A |
| | | | | | | I/II | | | |
| 8 | FFFFF.8 | FFFFF.8 | EDTGQDMLALFLRTNRQAAK | 201 | 20 | Class | Y | Y | Clear |
| | | | | | | I/II | | | |
| 9 | FFFFF.9 | FFFFF.9 | YGRTVVPFLVPGTSQLGQ | 202 | 18 | Class | Y | Y | Clear |
| | | | | | | I/II | | | |
| 10 | FFFFF.10 | FFFFF.10 | YIHTSVSQDFSQSVPGTTSSPL | 203 | 22 | Class | Y | Y | Clear |
| | | | | | | I/II | | | |
| 11 | FFFFF.11 | FFFFF.11 | VVVSSLLLQEEELLAGGKPGADG | 204 | 23 | Class | Y | N | N/A |
| | | | | | | I/II | | | |
| 12 | FFFFF.12 | FFFFF.12 | LSNQQPGLMVSFSLRLFPLFV | 205 | 21 | Class | N/A | N/A | N/A |
| | | | | | | I/II | | | |
| 13 | FFFFF.13 | FFFFF.13 | PVDNSSAKIVLKISYASKVKE | 206 | 21 | Class | N/A | N/A | N/A |
| | | | | | | I/II | | | |
| 14 | FFFFF.14 | FFFFF.14 | QIMLRSGVDLSVTDKREWRP | 207 | 20 | Class | Y | N | N/A |
| | | | | | | I/II | | | |
| 15 | FFFFF.15 | FFFFF.15 | RNQHQRLLKNMGAHLVVLDLLQIPYE | 208 | 26 | Class | N/A | N/A | N/A |
| | | | | | | I/II | | | |
| 16 | FFFFF.16 | FFFFF.16 | QPASAAKCTGDFSPSLEKLVKSGNPL | 209 | 30 | Class | N/A | N/A | N/A |
| | | | QPVS | | | I/II | | | |
| 17 | FFFFF.17 | FFFFF.17 | TPSAPEGYDLKIGLFLAPRRGSLPD | 210 | 25 | Class | Y | Y | Clear |
| | | | | | | I/II | | | |
| 18 | FFFFF.18 | FFFFF.18 | MILASPRYVDQVTEFLQQKLK | 211 | 21 | Class | Y | Y | Clear |
| | | | | | | I/II | | | |
| 19 | FFFFF.19 | FFFFF.19 | VGRAWDAITDHLVGLCISKSP | 212 | 21 | Class | Y | Y | Clear |
| | | | | | | I/II | | | |
| 20 | FFFFF.20 | FFFFF.20 | FLSPGQLLQEPRTSLLIINNT | 213 | 21 | Class | Y | Y | Cloudy |
| | | | | | | I/II | | | |
| 21 | FFFFF.21 | FFFFF.21 | RSTTKSPGPSRHSKSPASTSSVN | 214 | 23 | Class | Y | Y | Clear |
| | | | | | | I/II | | | |
| 22 | FFFFF.22 | FFFFF.22 | KLESTVGSPKKPLSDLGKLS | 215 | 20 | Class | Y | Y | Clear |
| | | | | | | I/II | | | |
| 23 | FFFFF.23 | FFFFF.23 | GRPRMMGTGLSPYPEHLTSPLSPAQ | 216 | 25 | Class | Y | N | N/A |
| | | | | | | I/II | | | |
| 24 | FFFFF.24 | FFFFF.24 | PLNPPASTAFSQEPHSGSPA | 217 | 20 | Class | Y | Y | Clear |
| | | | | | | I/II | | | |
| 25 | FFFFF.25 | FFFFF.25 | AAFVTPDQKYSMDNTLHTPTPFKNAL | 218 | 27 | Class | Y | N | N/A |
| | | | E | | | I/II | | | |
| 26 | FFFFF.26 | FFFFF.26 | RNLYISGFSLCFWLVLRRLVT | 219 | 21 | Class | Y | Y | Cloudy |
| | | | | | | I/II | | | |
| 27 | FFFFF.27 | FFFFF.27 | NAQLVVMILDGQNMAEDEF | 220 | 19 | Class | Y | N/A | N/A |
| | | | | | | I/II | | | |
| 28 | FFFFF.28 | FFFFF.28 | RVLCVGTLRLHELIKLTA | 221 | 18 | Class | Y | N/A | N/A |
| | | | | | | I/II | | | |
| 29 | FFFFF.29 | FFFFF.29 | HKDAVTCVNFSSSGHLLASGSR | 222 | 22 | Class | Y | Y | Clear |
| | | | | | | I/II | | | |
| 30 | FFFFF.30 | FFFFF.30 | LGNLAQFWECCLSSSGDADGESF | 223 | 23 | Class | N/A | N/A | N/A |
| | | | | | | I/II | | | |
| 31 | FFFFF.31 | FFFFF.31 | HIAGTSGFSLSFHSTVIN | 224 | 18 | Class | N/A | N/A | N/A |
| | | | | | | I/II | | | |
| 32 | FFFFF.32 | FFFFF.32 | LPTIKYLTLRLQDYLSLSHLVVYVPS | 225 | 26 | Class | N/A | N/A | N/A |
| | | | | | | I/II | | | |
| 33 | FFFFF.33 | FFFFF.33 | RLVHSGSGCRSPFLGSDLTFATRTGS | 226 | 28 | Class | N/A | N/A | N/A |
| | | | RQ | | | I/II | | | |
| 34 | FFFFF.34 | FFFFF.34 | PPKSPGPHSEKEDEAEPSTVPG | 227 | 22 | Class | Y | N | N/A |
| | | | | | | I/II | | | |
| 35 | FFFFF.35 | FFFFF.35 | PFLHTVSKTRLFEYLRLTSL | 228 | 20 | Class | Y | Y | Semi- |
| | | | | | | I/II | | | Cloudy |
| 36 | FFFFF.36 | FFFFF.36 | TGQDMLAL | 229 | 8 | Class I | Y | Y | Clear |
| 37 | FFFFF.37 | FFFFF.37 | YKTDLHSL | 230 | 8 | Class I | Y | Y | Clear |
| 38 | FFFFF.38 | FFFFF.38 | LAQFWECCL | 231 | 9 | Class I | Y | Y | Cloudy |
| 39 | FFFFF.39 | FFFFF.39 | SLLLQEEEL | 232 | 9 | Class I | Y | Y | Cloudy |
| 40 | FFFFF.40 | FFFFF.40 | CTGDFSPSL | 233 | 9 | Class I | Y | N | N/A |
| 41 | FFFFF.41 | FFFFF.41 | AQFWECCL | 234 | 8 | Class I | Y | Y | Cloudy |
| 42 | FFFFF.42 | FFFFF.42 | LLLQEEEL | 235 | 8 | Class I | Y | Y | Clear |
| 43 | FFFFF.43 | FFFFF.43 | TGDFSPSL | 236 | 8 | Class I | Y | Y | Semi- |
| | | | | | | | | | Cloudy |
| 44 | FFFFF.44 | FFFFF.44 | QVLESSSL | 237 | 8 | Class I | Y | N | N/A |
| 45 | FFFFF.45 | FFFFF.45 | NSLTSSVL | 238 | 8 | Class I | Y | Y | Clear |
| 46 | FFFFF.46 | FFFFF.46 | NSSAKIVL | 239 | 8 | Class I | Y | Y | Semi- |
| | | | | | | | | | Cloudy |
| 47 | FFFFF.47 | FFFFF.47 | NSASMKQAL | 240 | 9 | Class I | Y | Y | Clear |
| 48 | FFFFF.48 | FFFFF.48 | AIQVLESSSL | 241 | 10 | Class I | Y | Y | Semi- |
| | | | | | | | | | Cloudy |
| 49 | FFFFF.49 | FFFFF.49 | LGIGGLQDL | 242 | 9 | Class I | Y | Y | Clear |
| 50 | FFFFF.50 | FFFFF.50 | LSPYPEHL | 243 | 8 | Class I | Y | Y | Clear |
| 51 | FFFFF.51 | FFFFF.51 | AKFVAAWTLKAAA | 244 | 13 | PADRE | Y | Y | Clear |
| 52 | FFFFF.51 | FFFFF.51-1 | AKFVAAWTLKAAA | 245 | 13 | PADRE | Y | N/A | N/A |

TABLE 25
Peptide Synthesis Engineering Run Batch for Sample GGGGG

| | Peptide | Peptide | | SEQ | | Peptide | Final | Passed | Stability |
|---|---|---|---|---|---|---|---|---|---|
| No. | ID | Lot | Peptide Sequence | ID NO: | Length | Class | Lyo | QC | |
| 1 | GGGGG.1 | GGGGG.1 | SPRVAPGSAPPWPALRSLLH | 246 | 20 | Class | Y | Y | Clear |
| | | | | | | I/II | | | |
| 2 | GGGGG.2 | GGGGG.2 | VLGTSAPGSSRLAAVDLGG | 247 | 19 | Class | Y | Y | Clear |
| | | | | | | I/II | | | |
| 3 | GGGGG.3 | GGGGG.3 | ARPPGGSGPLRVLIPDLQL | 248 | 19 | Class | Y | Y | Clear |
| | | | | | | I/II | | | |
| 4 | GGGGG.4 | GGGGG.4 | PSAPQQEGVASKEKEEV | 249 | 17 | Class | Y | Y | Clear |
| | | | | | | I/II | | | |
| 5 | GGGGG.5 | GGGGG.5 | TSQAYNALTLVVTSCKNFKVRI | 250 | 22 | Class | N/A | N/A | N/A |
| | | | | | | I/II | | | |
| 6 | GGGGG.6 | GGGGG.6 | GENSVSSSPSASSTAALNTAAA | 251 | 22 | Class | N/A | N/A | N/A |
| | | | | | | I/II | | | |
| 7 | GGGGG.7 | GGGGG.7 | FETTTGFDPHSGTPLSDHEAL | 252 | 21 | Class | Y | Y | Clear |
| | | | | | | I/II | | | |
| 8 | GGGGG.8 | GGGGG.8 | FQSLCQAPPLLKDKVLTALE | 253 | 20 | Class | Y | Y | Clear |
| | | | | | | I/II | | | |
| 9 | GGGGG.9 | GGGGG.9 | VVPGNVTLSVVGSTSVPLSS | 254 | 20 | Class | N/A | N/A | N/A |
| | | | | | | I/II | | | |
| 10 | GGGGG.10 | GGGGG.10 | RPGEDPSLHGIVKEQL | 255 | 16 | Class | Y | Y | Clear |
| | | | | | | I/II | | | |
| 11 | GGGGG.11 | GGGGG.11 | SPAESCDLLGAIQTCIRKSLG | 256 | 21 | Class | Y | Y | Clear |
| | | | | | | I/II | | | |
| 12 | GGGGG.12 | GGGGG.12 | MKMASFLAFLLLNFHVCLLLLQ | 257 | 25 | Class | N/A | N/A | N/A |
| | | | LLM | | | I/II | | | |
| 13 | GGGGG.13 | GGGGG.13 | SENQQPGAPNTPTHPAPPGLH | 258 | 21 | Class | N/A | N/A | N/A |
| | | | | | | I/II | | | |
| 14 | GGGGG.14 | GGGGG.14 | HLINYQDDAELATHALPELTKL | 259 | 29 | Class | Y | Y | Clear |
| | | | LNDEDPV | | | I/II | | | |
| 15 | GGGGG.15 | GGGGG.15 | LIVENVHFQAHKALLAA | 260 | 17 | Class | Y | Y | Semi |
| | | | | | | I/II | | | Cloudy |
| 16 | GGGGG.16 | GGGGG.16 | STAPAEATLPKPGEAEAP | 261 | 18 | Class | Y | Y | Clear |
| | | | | | | I/II | | | |
| 17 | GGGGG.17 | GGGGG.17 | PYSGLGGVGDPYAPLMVLMCR | 262 | 26 | Class | Y | Y | Semi |
| | | | VCLED | | | I/II | | | Cloudy |
| 18 | GGGGG.18 | GGGGG.18 | DFYLRGAVALSVRPIS | 263 | 16 | Class | N/A | N/A | N/A |
| | | | | | | I/II | | | |
| 19 | GGGGG.19 | GGGGG.19 | TDVDPQSAVMQEEIF | 264 | 15 | Class | N/A | N/A | N/A |
| | | | | | | I/II | | | |
| 20 | GGGGG.20 | GGGGG.20 | NSLQNQALQTLQERLHEADAT | 265 | 21 | Class | Y | Y | Clear |
| | | | | | | I/II | | | |
| 21 | GGGGG.21 | GGGGG.21 | GAEDSIDSPSACPLSTGCPAL | 266 | 21 | Class | Y | Y | Clear |
| | | | | | | I/II | | | |
| 22 | GGGGG.22 | GGGGG.22 | RFIGPLPREGSVGSTSDYVSQS | 267 | 22 | Class | Y | Y | Clear |
| | | | | | | I/II | | | |
| 23 | GGGGG.23 | GGGGG.23 | IQSIYGGLPKVPAKPKEPTIPH | 268 | 22 | Class | Y | Y | Clear |
| | | | | | | I/II | | | |
| 24 | GGGGG.24 | GGGGG.24 | FWGILGFPALYTHLPAFLEWTL | 269 | 26 | Class | Y | N | N/A |
| | | | CLLS | | | I/II | | | |
| 25 | GGGGG.25 | GGGGG.25 | VDLKFPASVPTGAQDLISKL | 270 | 20 | Class | Y | Y | Clear |
| | | | | | | I/II | | | |
| 26 | GGGGG.26 | GGGGG.26 | LAPGQPFLSSQGSLCI | 271 | 16 | Class | N/A | N/A | N/A |
| | | | | | | I/II | | | |
| 27 | GGGGG.27 | GGGGG.27 | RVGDLSPKQKEALAKPEA | 272 | 18 | Class | Y | Y | Clear |
| | | | | | | I/II | | | |
| 28 | GGGGG.28 | GGGGG.28 | LAVRWFFAHSSDSQEALMV | 273 | 19 | Class | Y | N | N/A |
| | | | | | | I/II | | | |
| 29 | GGGGG.29 | GGGGG.29 | YSGIQESSSASPLSIKKCPI | 274 | 20 | Class | Y | Y | Clear |
| | | | | | | I/II | | | |
| 30 | GGGGG.30 | GGGGG.30 | LIKPPAHTSAILTVLR | 275 | 16 | Class | Y | Y | Clear |
| | | | | | | I/II | | | |
| 31 | GGGGG.31 | GGGGG.31 | MSYELKCAQELSQKQDG | 276 | 17 | Class | Y | Y | Clear |
| | | | | | | I/II | | | |
| 32 | GGGGG.32 | GGGGG.32 | QVHQCSVLLVATGLSVP | 277 | 17 | Class | N/A | N/A | N/A |
| | | | | | | I/II | | | |
| 33 | GGGGG.33 | GGGGG.33 | RSLTLEPDPIVVPGNVTLSWG | 278 | 22 | Class | Y | Y | Clear |
| | | | | | | I/II | | | |
| 34 | GGGGG.34 | GGGGG.34 | LDRQHVQHQLLVILKELRK | 279 | 19 | Class | Y | Y | Clear |
| | | | | | | I/II | | | |
| 35 | GGGGG.35 | GGGGG.35 | TVDMLQCLRFPGLALPHTRAPS | 280 | 25 | Class | Y | Y | Clear |
| | | | PLG | | | I/II | | | |
| 36 | GGGGG.36 | GGGGG.36 | SAPGSSRLAAV | 281 | 11 | Class | Y | Y | Clear |
| | | | | | | I | | | |
| 37 | GGGGG.37 | GGGGG.37 | VALSVRPI | 282 | 8 | Class | Y | Y | Clear |
| | | | | | | I | | | |
| 38 | GGGGG.38 | GGGGG.38 | SAVMQEEI | 283 | 8 | Class | Y | Y | Cloudy |
| | | | | | | I | | | |
| 39 | GGGGG.39 | GGGGG.39 | AAIQEKKEI | 284 | 9 | Class | Y | Y | Clear |
| | | | | | | I | | | |
| 40 | GGGGG.40 | GGGGG.40 | VSPDIFMQSHL | 285 | 11 | Class | Y | Y | Clear |
| | | | | | | I | | | |
| 41 | GGGGG.41 | GGGGG.41 | QAYNALTL | 286 | 8 | Class | Y | Y | Clear |
| | | | | | | I | | | |
| 42 | GGGGG.42 | GGGGG.42 | RVLIPDLQL | 287 | 9 | Class | Y | Y | Clear |
| | | | | | | I | | | |
| 43 | GGGGG.43 | GGGGG.43 | APGSSRLA | 288 | 8 | Class | Y | Y | Clear |
| | | | | | | I | | | |
| 44 | GGGGG.44 | GGGGG.44 | STAPAEATL | 289 | 9 | Class | Y | Y | Clear |
| | | | | | | I | | | |
| 45 | GGGGG.45 | GGGGG.45 | GALPVASPASL | 290 | 11 | Class | Y | Y | Clear |
| | | | | | | I | | | |
| 46 | GGGGG.46 | GGGGG.46 | SAPGSSRLAA | 291 | 10 | Class | Y | Y | Clear |
| | | | | | | I | | | |
| 47 | GGGGG.47 | GGGGG.47 | TSAPGSSRLAA | 292 | 11 | Class | Y | Y | Clear |
| | | | | | | I | | | |
| 48 | GGGGG.48 | GGGGG.48 | SLCQAPPL | 293 | 8 | Class | Y | Y | Clear |
| | | | | | | I | | | |
| 49 | GGGGG.49 | GGGGG.49 | MSYELKCAQEL | 294 | 11 | Class | Y | Y | Clear |
| | | | | | | I | | | |
| 50 | GGGGG.50 | GGGGG.50 | LAPGQPFL | 295 | 8 | Class | y | Y | Clear |
| | | | | | | I | | | |
| 51 | GGGGG.51 | GGGGG.51 | AKFVAAWTLKAAA | 296 | 13 | PADRE | Y | Y | Clear |
| 52 | GGGGG.52 | GGGGG.51-1 | AKFVAAWTLKAAA | 297 | 13 | PADRE | N/A | N/A | N/A |

Peptides that were successfully manufactured as determined by passing quality control criteria (appearance, identity, peptide content, peptide purity, acetate content, TFA content, residual organic solvents), were then ingested into a pool optimization algorithm.

Briefly, this algorithm distributed the selected peptide to 4 pools, optimizing distribution so that each pool contains peptides with both high MHC class I and class II scores. After initial exclusion of sequences which contain more than two cysteines to avoid multimerization during pool formulation, an optimization algorithm identified the best combination of approximately 14 long vaccine peptides and 6 short peptides to be combined into individual vaccine peptide pools. If the initial peptide prediction or manufacturing yielded less than 14 long vaccine peptides, the long—to—short peptide ratio may be changed to accommodate the target of four pools with five peptides each. Similarly, if a vaccine peptide pool was composed of less than three (3) long peptides, which extrapolates to a low chance of CD4 engagement, one of the pool peptides was chosen to be PADRE.

Several proposed pool compositions were then communicated to the peptide manufacturer, and vaccine peptide pool formulation is performed:

Groups of peptides are selected and pooled. Up to four (4) pools with not more than (NMT) five (5) peptides in each pool were prepared. Each peptide was dissolved in 5.538% (v/v) DMSO followed by 0.9% NaCl solution, at a concentration of each peptide at 0.4158 mg/mL. Peptides which were visibly dissolved were accepted for that group's pool. Upon successful pooling of all peptides within a group, the pool was filtered through a 0.2 μm Nylon filter. Peptides were shipped in sterile tubes and labeled with Pool name, lot #, components, manufacturing data, quantity/concentration and storage conditions.

### 1.5. Formulation of Peptide Vaccine

To formulate a personalized vaccine, peptide pools were admixed with Poly ICLC as adjuvant using the following procedure:

Remove one peptide vial each per pool from the freezer. The following steps are performed for each of the peptide pools.

Thaw the peptide pools for 20 to 30 min at room temperature. The peptide solution contains DMSO and may require slight warming (hand-warm) and/or agitation for complete thawing. The substance is expected to be a clear, colorless solution. If precipitates form, the solution may be vortexed to resolve precipitates.

While thawing, prepare and label syringes for steps 4-9 (see Table 26). (A) Two (2) mixing syringes per pool, labeled “M1-2 [A-D]” (denoting pools): (i) Syringe 1: 10 mL (or appropriately sized) and (ii) Syringe 2: 1 mL (or appropriately sized); and (B) One (1) administration syringe per pool, labeled “administration syringe [A-D]” (denoting pools).

TABLE 26
List of syringes used for preparation and administration.

| | Suggested | |
|---|---|---|
| | Syringe | |
| Syringe Label^{a} | Volume^{b} | Syringe Use |
| M1_[pool name] | 10 mL | Aspiration of peptide pool, sterile filtration |
| M2_[pool name] | 1 mL | Aspiration Poly ICLC, transfer into M1 |
| A1_[pool name] | 1 mL | Receiving Syringe for final drug product, |
| | | Administration |

^{a}All syringes are to be prepared once each per peptide pool
^{b}Use suggested volume syringe or appropriate size

### Additional Material Required (Per Peptide Pool)

1 (one) guarded female to female Luer adaptor

1 (one) 3″ aspiration needle to transfer peptide pool into mixing syringe M1

1 (one) sterile low-protein binding syringe filter, non-pyrogenic. Pore size 0.22 μm. (e.g. Pall DMSO-Safe Aerodisc Syringe Filter, #4433, or Millex-GV 0.22 μm PVDF, 33 mm, gamma sterilized. Millipore, #SLGVM33RS)

3 (three) Sterile Hypodermic needle appropriate gauge

1 (one) Spiros Closed System Drug-Transfer Device (CSTD) for IM injection [ICU Medical, #SH2000SC-10]

Sterile filter the content of each vial: Using the 10mL syringe with Luer Lock (mixing syringe 1, M1), connected to the 0.22 μm sterile filter, slide the 3″ aspiration needle on the assembly, and remove 2mL peptide pool from the vial.

Prepare Poly ICLC solution. Using a 1mL syringe (mixing syringe 2, M2) and appropriately sized needle, aspirate 0.760 mL of Poly ICLC under sterile conditions. Poly ICLC is a white opalescent solution. Downstream steps may lead to an opalescent product, which is acceptable.

Remove the needles from mixing syringe 1 (M1) and mixing syringe 2 (M2), as well as sterile filter from mixing syringe 1 (M1).

Connect Poly ICLC mixing syringe 2 (M2) with the peptide pool mixing syringe 1 (M1) via a female-to-female Luer Lock guarded connector

Transfer Poly ICLC mixing syringe 2 (M2) into mixing syringe 1 (M1) and mix diligently between both syringes. Bubbles may form in the product. Tapping the syringe may help to collect bubbles. The resultant mixture is the personalized vaccine.

1. A method, comprising:
a) obtaining sequence data from the tumor, wherein the sequence data is used to obtain data representing a polypeptide sequence of one or more tumor-specific neoantigens;
b) obtaining data representing MHC molecule polypeptide sequence data from the subject;
c) inputting the data representing the polypeptide sequence(s) and the data representing the MHC molecule(s) polypeptide sequence(s) into a machine-learning platform to generate a numerical probability score that the one or more tumor-specific neoantigens will elicit an immune response in the subject;
d) quantifying RNA expression of the one or more tumor-specific neoantigens in a tumor to identify one or more tumor-specific neoantigens that express an amount of the one or more tumor-specific neoantigens sufficient to elicit an immune response in the subject;
e) calculating a tumor-specific neoantigen score based on steps c) and d) for the one or more tumor-specific neoantigens; and
f) selecting one or more tumor-specific neoantigens based on the tumor-specific score for formulation of a subject-specific immunogenic composition.

2. The method of claim 1, further comprising:
g) forming a subject-specific immunogenic composition comprising the one or more selected tumor-specific neoantigens; and
h) administering the immunogenic composition to the subject.

3. The method of claim 1, further comprising, before step e), sequencing tumor clones of the tumor to identify one or more tumor-specific neoantigens that represent a sufficient fraction of the tumor.

4. The method of claim 3, wherein the tumor-specific neoantigen score is calculated based on steps c), d), and the sequencing the tumor clones.

5. The method of claim 1, wherein the polypeptide sequence of the one or more tumor-specific neoantigens is from short polypeptides.

6. (canceled)

7. The method of claim 1, wherein the polypeptide sequence of the one or more tumor-specific neoantigens is from long polypeptides.

8-13. (canceled)

14. The method of claim 1, wherein a tumor-specific neoantigen with a higher numerical probability score relative to a lower numerical probability score indicates that the tumor-specific neoantigen will elicit a greater immune response in the subject.

15. (canceled)

16. The method of claim 1, wherein the quantifying the RNA expression comprises measuring mRNA expression.

17. The method of claim 3, wherein the one or more tumor-specific neoantigens represent at least about 1% of the tumor.

18. The method of claim 3, wherein the one or more tumor-specific neoantigens represent at least about 5% of the tumor.

19. The method of claim 1, wherein the sequence data is nucleotide sequence data.

20. The method of claim 1, wherein the sequence data is polypeptide sequence data.

21. (canceled)

22. The method of claim 1, wherein at least about 10 tumor-specific neoantigens are selected to formulate the subject-specific immunogenic composition.

23. The method of claim 22, wherein at least about 20 tumor-specific neoantigens are selected to formulate the subject-specific immunogenic composition.

24-25. (canceled)

26. The method of claim 1, wherein the immunogenic composition comprises one or more tumor-specific neoantigens encoded by short polypeptides.

27. The method of claim 1, wherein the immunogenic composition comprises one or more tumor-specific neoantigens encoded by long polypeptides.

28. (canceled)

29. The method of claim 1, further comprising identifying one or more tumor-specific neoantigens that induce an autoimmune response to normal tissue.

30. The method of claim 29, wherein, in step e), the one or more tumor-specific neoantigens that induce an autoimmune response to normal tissue are not selected for the immunogenic composition.

31. The method of claim 29, wherein the one or more tumor-specific neoantigens that induce an autoimmune response to normal tissue has a lower tumor-specific neoantigen score relative to a tumor-specific neoantigen that does not induce an autoimmune response.

32. The method of claim 1, wherein the tumor is from melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer.

33. The method of claim 32, wherein the tumor is from a cancer selected from the group consisting of a melanoma, breast cancer, lung cancer, and bladder cancer.

34-41. (canceled)

42. The method of claim 1, further comprising formulating an immunogenic composition comprising the selected one or more tumor-specific neoantigens.

43. An immunogenic composition comprising one or more tumor-specific neoantigens selected by performing the method of claim 1.

44. The immunogenic composition of claim 43, wherein the immunogenic composition comprises a nucleotide sequence, a polypeptide sequence, RNA, DNA, a cell, a plasmid, a vector, a dendritic cell, or a synthetic long peptide.

45. The immunogenic composition of claim 43, further comprising an adjuvant.

46. The method of claim 1, wherein the sequence data is whole exome sequence data, RNA sequence data, whole genome sequence data or combinations thereof.

47. (canceled)

48. A method for treating cancer in a subject in need thereof, comprising:
a) obtaining sequence data from the tumor, wherein the sequence data is used to obtain data representing a polypeptide sequence of one or more tumor-specific neoantigens;
b) obtaining data representing MHC molecule polypeptide sequence data from the subject;
c) inputting the data representing the polypeptide sequence(s) and the data representing the MHC molecule(s) polypeptide sequence(s) into a machine-learning platform to generate a numerical probability score that the one or more tumor-specific neoantigens will elicit an immune response in the subject;
d) quantifying RNA expression of the one or more tumor-specific neoantigens in a tumor to identify one or more tumor-specific neoantigens that express an amount of the one or more tumor-specific neoantigens sufficient to elicit an immune response in the subject;
e) calculating a tumor-specific neoantigen score based on steps c) and d) for the one or more tumor-specific neoantigens; and
f) selecting one or more tumor-specific neoantigens based on the tumor-specific score for formulation of a subject-specific immunogenic composition.
g) forming a subject-specific immunogenic composition comprising the one or more selected tumor-specific neoantigens; and
h) administering the immunogenic composition to the subject.


SEQUENCE LISTING (297 sequences)

SEQ ID NO: 1 - DNA, 16 bp, Homo sapiens
gtaggtgctgtcacat

SEQ ID NO: 2 - protein, 15 aa, Homo sapiens
QDSNTSALPLVSLFF

SEQ ID NO: 3 - protein, 16 aa, Homo sapiens
YIKYKVEQVGDMIDRL

SEQ ID NO: 4 - protein, 16 aa, Homo sapiens
GQRRDRLLTVQALSGL

SEQ ID NO: 5 - protein, 17 aa, Homo sapiens
YIKYKVEQVGDMIDRLF

SEQ ID NO: 6 - protein, 22 aa, Homo sapiens
EGIKMSKPKWPLEDEISKPEVP

SEQ ID NO: 7 - protein, 16 aa, Homo sapiens
QFDQLYRTKPGMTMSC

SEQ ID NO: 8 - protein, 15 aa, Homo sapiens
GDVAVKMLTPQQLQA

SEQ ID NO: 9 - protein, 20 aa, Homo sapiens
RLEQAWMNEKLAPELLESKP

SEQ ID NO: 10 - protein, 22 aa, Homo sapiens
NEVLAASNLDPAALRLVSRKLE

SEQ ID NO: 11 - protein, 22 aa, Homo sapiens
GARRWRKLYRANSHLFQAKRFN

SEQ ID NO: 12 - protein, 22 aa, Homo sapiens
SQGIIYRDLKLENIMLSSQGHI

SEQ ID NO: 13 - protein, 21 aa, Homo sapiens
GSEPTGAYSIKGFFADYEIPN

SEQ ID NO: 14 - protein, 16 aa, Homo sapiens
YRAPEIILGLLFCEAI

SEQ ID NO: 15 - protein, 16 aa, Homo sapiens
APPTRLAPPQRPPPPS

SEQ ID NO: 16 - protein, 21 aa, Homo sapiens
LQMLCPQEAPQILSRLEAVRR

SEQ ID NO: 17 - protein, 18 aa, Homo sapiens
SLFQSFETWHRGDALSRL

SEQ ID NO: 18 - protein, 19 aa, Homo sapiens
SGQQWYEVEHTERIKNCLN

SEQ ID NO: 19 - protein, 17 aa, Homo sapiens
AVVSYVVADMKEMLPRS

SEQ ID NO: 20 - protein, 16 aa, Homo sapiens
GQLFPYRARIEIKNKF

SEQ ID NO: 21 - protein, 15 aa, Homo sapiens
ARPEAQSRSSPTLES

SEQ ID NO: 22 - protein, 19 aa, Homo sapiens
TYPEADSFPSWAAAHRKGS

SEQ ID NO: 23 - protein, 20 aa, Homo sapiens
FELLDQGELEKLNAELGLEP

SEQ ID NO: 24 - protein, 22 aa, Homo sapiens
EKLKSWGGSLLGPWLSSGLKPL

SEQ ID NO: 25 - protein, 22 aa, Homo sapiens
YHKQQEELDAFKKTIEESEETL

SEQ ID NO: 26 - protein, 22 aa, Homo sapiens
HGAWLPELPSLPSNGDPPAICE

SEQ ID NO: 27 - protein, 16 aa, Homo sapiens
QDDFTALGQMTEKEHG

SEQ ID NO: 28 - protein, 19 aa, Homo sapiens
RDILELGGPEEDAASGTIS

SEQ ID NO: 29 - protein, 22 aa, Homo sapiens
GPLGTGAFEAPGFNTTTATLGF

SEQ ID NO: 30 - protein, 21 aa, Homo sapiens
FGALHEDANKVIKPTSSNTAQ

SEQ ID NO: 31 - protein, 22 aa, Homo sapiens
KSGDAAIVDMVPSKPMCVESFS

SEQ ID NO: 32 - protein, 22 aa, Homo sapiens
REDDDESAGENPLEEEEEQPAP

SEQ ID NO: 33 - protein, 22 aa, Homo sapiens
SWRVTVEPQDSNTSALPLVSLF

SEQ ID NO: 34 - protein, 20 aa, Homo sapiens
PASSQAGTVTLYGPTSSVAL

SEQ ID NO: 35 - protein, 22 aa, Homo sapiens
DGADTSVFSNNVVFVTGNYVLD

SEQ ID NO: 36 - protein, 22 aa, Homo sapiens
WLRRTKPSKHQYQICLAIDDSS

SEQ ID NO: 37 - protein, 22 aa, Homo sapiens
VPGIPTAVPAGPTVPTVPTVEA

SEQ ID NO: 38 - protein, 20 aa, Homo sapiens
PVKSQSKSNTFLPPLPFKRG

SEQ ID NO: 39 - protein, 17 aa, Homo sapiens
GWTTMRRACGARTLSRA

SEQ ID NO: 40 - protein, 22 aa, Homo sapiens
MGDPGSLQPKSAETMNNIDRLR

SEQ ID NO: 41 - protein, 16 aa, Homo sapiens
DPRLVIFSGCATRLFE

SEQ ID NO: 42 - protein, 15 aa, Homo sapiens
KHSVKVTNCFSVPHN

SEQ ID NO: 43 - protein, 22 aa, Homo sapiens
EKAHTLILQPPSKKGRQCEWRS

SEQ ID NO: 44 - protein, 9 aa, Homo sapiens
YIKYKVEQV

SEQ ID NO: 45 - protein, 10 aa, Homo sapiens
LEDEISKPEV

SEQ ID NO: 46 - protein, 8 aa, Homo sapiens
YRANSHLF

SEQ ID NO: 47 - protein, 9 aa, Homo sapiens
SIKGFFADY

SEQ ID NO: 48 - protein, 9 aa, Homo sapiens
QRRDRLLTV

SEQ ID NO: 49 - protein, 9 aa, Homo sapiens
YVVADMKEM

SEQ ID NO: 50 - protein, 17 aa, Homo sapiens
AMETALVTLILLQIGKS

SEQ ID NO: 51 - protein, 22 aa, Homo sapiens
KGNIAQMYHAFGKKREFNAFVL

SEQ ID NO: 52 - protein, 20 aa, Homo sapiens
KYLTLRLQDYLSLSHLVVYV

SEQ ID NO: 53 - protein, 20 aa, Homo sapiens
GRAWDAITDHLVGLCISKSP

SEQ ID NO: 54 - protein, 22 aa, Homo sapiens
VGSEIAGGAGLGWVLPFTAGGF

SEQ ID NO: 55 - protein, 19 aa, Homo sapiens
GFRRVLCVGTLRLHELIKL

SEQ ID NO: 56 - protein, 18 aa, Homo sapiens
SAIQVLESSSLSLTDSLN

SEQ ID NO: 57 - protein, 16 aa, Homo sapiens
LPLLLFSRAPTVDPPR

SEQ ID NO: 58 - protein, 15 aa, Homo sapiens
LPLLLFSRAPTVDPP

SEQ ID NO: 59 - protein, 21 aa, Homo sapiens
AKCTGDFSPSLEKLVKSGNPL

SEQ ID NO: 60 - protein, 22 aa, Homo sapiens
GESNSLTSSVLYPTASLVSQNE

SEQ ID NO: 61 - protein, 18 aa, Homo sapiens
GAKGEVEADGIPGFPGLP

SEQ ID NO: 62 - protein, 21 aa, Homo sapiens
PVDNSSAKIVLKISYASKVKE

SEQ ID NO: 63 - protein, 22 aa, Homo sapiens
VVVSSLLLQEEELLAGGKPGAD

SEQ ID NO: 64 - protein, 21 aa, Homo sapiens
ARKCLAKQAEDAAREKSCKRG

SEQ ID NO: 65 - protein, 22 aa, Homo sapiens
RNLYISGFSLCFWLVLRRLVTL

SEQ ID NO: 66 - protein, 21 aa, Homo sapiens
KQKYLTVISNRRWLLEPIPRK

SEQ ID NO: 67 - protein, 21 aa, Homo sapiens
RFSAPLFLSHSHFLNADPVLA

SEQ ID NO: 68 - protein, 22 aa, Homo sapiens
WDHNAQLVVMILDGQNMAEDEF

SEQ ID NO: 69 - protein, 22 aa, Homo sapiens
AIWDGYLQQAGPFFIYFLMLII

SEQ ID NO: 70 - protein, 18 aa, Homo sapiens
ASHLEEYNERLELILKWI

SEQ ID NO: 71 - protein, 21 aa, Homo sapiens
KRELVNSASMKQALIASARRL

SEQ ID NO: 72 - protein, 16 aa, Homo sapiens
PLDPGGYFIIMDQKRF

SEQ ID NO: 73 - protein, 22 aa, Homo sapiens
LQEALTSRKAIPKKAQEKERHL

SEQ ID NO: 74 - protein, 22 aa, Homo sapiens
LNRMNGVMFSGNSPSYTERSNI

SEQ ID NO: 75 - protein, 19 aa, Homo sapiens
QRLALWEGPFKAHTKGSHQ

SEQ ID NO: 76 - protein, 21 aa, Homo sapiens
REYFGEKTDLKEKIDIGLPPP

SEQ ID NO: 77 - protein, 22 aa, Homo sapiens
PALEEEEAPQALSLLSLPPKKR

SEQ ID NO: 78 - protein, 20 aa, Homo sapiens
LSNRCQVFDCFQDTVSQHVV

SEQ ID NO: 79 - protein, 19 aa, Homo sapiens
DCYRVDIDQEADMQKESKE

SEQ ID NO: 80 - protein, 21 aa, Homo sapiens
RRRGGCEKLRAEPQAVLASGS

SEQ ID NO: 81 - protein, 17 aa, Homo sapiens
HFYKVIGVFIRAEDGLC

SEQ ID NO: 82 - protein, 20 aa, Homo sapiens
ALTLVFDDVQGYDLMGSVTL

SEQ ID NO: 83 - protein, 22 aa, Homo sapiens
MILASPRYVDQVTEFLQQKLKQ

SEQ ID NO: 84 - protein, 22 aa, Homo sapiens
NSTQDNNYGCVSLLEDSETRKN

SEQ ID NO: 85 - protein, 22 aa, Homo sapiens
EEEERKRRPPSLEPSTKVSEEA

SEQ ID NO: 86 - protein, 22 aa, Homo sapiens
AGGGPPPAPPLSAAQGPGGGGA

SEQ ID NO: 87 - protein, 22 aa, Homo sapiens
KLNDADPDDKFYCLRLFRHFYH

SEQ ID NO: 88 - protein, 22 aa, Homo sapiens
VQQKLEQLNQYSDFNNYLIFVL

SEQ ID NO: 89 - protein, 20 aa, Homo sapiens
TTVETLEKENSWYCPSCKQH

SEQ ID NO: 90 - protein, 15 aa, Homo sapiens
VDGDWVLHLPEALSA

SEQ ID NO: 91 - protein, 18 aa, Homo sapiens
RFRRLRQEREFLGLWGPE

SEQ ID NO: 92 - protein, 10 aa, Homo sapiens
METALVTLIL

SEQ ID NO: 93 - protein, 9 aa, Homo sapiens
SVLYPTASL

SEQ ID NO: 94 - protein, 10 aa, Homo sapiens
ALVTLILLQI

SEQ ID NO: 95 - protein, 11 aa, Homo sapiens
EADGIPGFPGL

SEQ ID NO: 96 - protein, 11 aa, Homo sapiens
GEVEADGIPGF

SEQ ID NO: 97 - protein, 10 aa, Homo sapiens
RLWTSENKSK

SEQ ID NO: 98 - protein, 19 aa, Homo sapiens
HEHWRFVLQHLVFLAAFVV

SEQ ID NO: 99 - protein, 22 aa, Homo sapiens
QSPARAREEENHSFLPLVHNII

SEQ ID NO: 100 - protein, 17 aa, Homo sapiens
EDEEEENIELKVTKPVQ

SEQ ID NO: 101 - protein, 21 aa, Homo sapiens
PELMPFRLTCQFINLMLPMKE

SEQ ID NO: 102 - protein, 22 aa, Homo sapiens
EEWQQLDPEQNITYRDVMLENY

SEQ ID NO: 103 - protein, 20 aa, Homo sapiens
DFKGRFKARPKLEELLAKLK

SEQ ID NO: 104 - protein, 22 aa, Homo sapiens
ASSQSMPWLVDLVQSSEGSLDV

SEQ ID NO: 105 - protein, 21 aa, Homo sapiens
RFFPPKSNKACHYHSYNGWNR

SEQ ID NO: 106 - protein, 21 aa, Homo sapiens
MYLAGYHCRNCNVREALQAWA

SEQ ID NO: 107 - protein, 15 aa, Homo sapiens
LVFCLLPSKDVQFLS

SEQ ID NO: 108 - protein, 19 aa, Homo sapiens
RRRQCDLVGVETCKSLESQ

SEQ ID NO: 109 - protein, 21 aa, Homo sapiens
FLRALKENKDQKEQAAKAERR

SEQ ID NO: 110 - protein, 9 aa, Homo sapiens
FVLQHLVFL

SEQ ID NO: 111 - protein, 9 aa, Homo sapiens
REEENHSFL

SEQ ID NO: 112 - protein, 11 aa, Homo sapiens
REEENHSFLPL

SEQ ID NO: 113 - protein, 9 aa, Homo sapiens
VLQHLVFLA

SEQ ID NO: 114 - protein, 9 aa, Homo sapiens
EENHSFLPL

SEQ ID NO: 115 - protein, 9 aa, Homo sapiens
SMPWLVDLV

SEQ ID NO: 116 - protein, 10 aa, Homo sapiens
RAREEENHSF

SEQ ID NO: 117 - protein, 9 aa, Homo sapiens
KARPKLEEL

SEQ ID NO: 118 - protein, 10 aa, Homo sapiens
FVLQHLVFLA

SEQ ID NO: 119 - protein, 9 aa, Homo sapiens
RLTCQFINL

SEQ ID NO: 120 - protein, 10 aa, Homo sapiens
QLDPEQNITY

SEQ ID NO: 121 - protein, 11 aa, Homo sapiens
NIELKVTKPVQ

SEQ ID NO: 122 - protein, 8 aa, Homo sapiens
REEENHSF

SEQ ID NO: 123 - protein, 10 aa, Homo sapiens
EEENHSFLPL

SEQ ID NO: 124 - protein, 10 aa, Homo sapiens
NIELKVTKPV

SEQ ID NO: 125 - protein, 8 aa, Homo sapiens
FVLQHLVF

SEQ ID NO: 126 - protein, 11 aa, Homo sapiens
DEEEENIELKV

SEQ ID NO: 127 - protein, 10 aa, Homo sapiens
EEEENIELKV

SEQ ID NO: 128 - protein, 11 aa, Homo sapiens
RAREEENHSFL

SEQ ID NO: 129 - protein, 10 aa, Homo sapiens
VLQHLVFLAA

SEQ ID NO: 130 - protein, 11 aa, Homo sapiens
EEEENIELKVT

SEQ ID NO: 131 - protein, 10 aa, Homo sapiens
QSMPWLVDLV

SEQ ID NO: 132 - protein, 9 aa, Homo sapiens
LTCQFINLM

SEQ ID NO: 133 - protein, 11 aa, Homo sapiens
EEENIELKVTK

SEQ ID NO: 134 - protein, 8 aa, Homo sapiens
VLQHLVFL

SEQ ID NO: 135 - protein, 10 aa, Homo sapiens
LLPSKDVQFL

SEQ ID NO: 136 - protein, 11 aa, Homo sapiens
EDEEEENIELK

SEQ ID NO: 137 - protein, 11 aa, Homo sapiens
QLDPEQNITYR

SEQ ID NO: 138 - protein, 9 aa, Homo sapiens
ALKENKDQK

SEQ ID NO: 139 - protein, 8 aa, Homo sapiens
KLEELLAK

SEQ ID NO: 140 - protein, 11 aa, Homo sapiens
AREEENHSFLP

SEQ ID NO: 141 - protein, 8 aa, Homo sapiens
KSNKACHY

SEQ ID NO: 142 - protein, 8 aa, Homo sapiens
PSKDVQFL

SEQ ID NO: 143 - protein, 9 aa, Homo sapiens
EEEENIELK

SEQ ID NO: 144 - protein, 13 aa, Homo sapiens
AKFVAAWTLKAAA

SEQ ID NO: 145 - protein, 13 aa, Homo sapiens
AKFVAAWTLKAAA

SEQ ID NO: 146 - protein, 25 aa, Artificial Sequence
  Description of Artificial Sequence Synthetic peptide
PASSQAGTVTLYGPTSSVALGFTSL

SEQ ID NO: 147 - protein, 21 aa, Artificial Sequence
  Description of Artificial Sequence Synthetic peptide
EDDDESAGENPLEEEEEQPAP

SEQ ID NO: 148 - protein, 18 aa, Artificial Sequence
  Description of Artificial Sequence Synthetic peptide
MSKPKWPLEDEISKPEVP

SEQ ID NO: 149 - protein, 28 aa, Artificial Sequence
  Description of Artificial Sequence Synthetic peptide
NQPKIGGPLGTGAFEAPGFNTTTATLGF

SEQ ID NO: 150 - protein, 20 aa, Artificial Sequence
  Description of Artificial Sequence Synthetic peptide
FGALHEDANKVIKPTSSNTA

SEQ ID NO: 151 - protein, 28 aa, Artificial Sequence
  Description of Artificial Sequence Synthetic peptide
KNPQMGDPGSLQPKSAETMNNIDRLRME

SEQ ID NO: 152 - protein, 20 aa, Artificial Sequence
  Description of Artificial Sequence Synthetic peptide
LQMLCPQEAPQILSRLEAVR

SEQ ID NO: 153 - protein, 23 aa, Artificial Sequence
  Description of Artificial Sequence Synthetic peptide
YVMVVMSDSSIPSAATLINIRNA

SEQ ID NO: 154 - protein, 20 aa, Artificial Sequence
  Description of Artificial Sequence Synthetic peptide
SSCMGGMNQRPILTIITLED

SEQ ID NO: 155 - protein, 16 aa, Artificial Sequence
  Description of Artificial Sequence Synthetic peptide
YRAPEIILGLLFCEAI

SEQ ID NO: 156 - protein, 17 aa, Artificial Sequence
  Description of Artificial Sequence Synthetic peptide
DPRLVIFSGCATRLFEA

SEQ ID NO: 157 - protein, 15 aa, Artificial Sequence
  Description of Artificial Sequence Synthetic peptide
ARPEAQSRSSPTLES

SEQ ID NO: 158 - protein, 16 aa, Artificial Sequence
  Description of Artificial Sequence Synthetic peptide
DGADTSVFSNNVVFVT

SEQ ID NO: 159 - protein, 16 aa, Artificial Sequence
  Description of Artificial Sequence Synthetic peptide
GQRRDRLLTVQALSGL

SEQ ID NO: 160 - protein, 19 aa, Artificial Sequence
  Description of Artificial Sequence Synthetic peptide
WLPELPSLPSNGDPPAICE

SEQ ID NO: 161 - protein, 21 aa, Artificial Sequence
  Description of Artificial Sequence Synthetic peptide
LLGTVDKHSVKVTNCFSVPHN

SEQ ID NO: 162 - protein, 28 aa, Artificial Sequence
  Description of Artificial Sequence Synthetic peptide
KDKIWLRRTKPSKHQYQICLAIDDSSSM

SEQ ID NO: 163 - protein, 28 aa, Artificial Sequence
  Description of Artificial Sequence Synthetic peptide
QELSPEKLKSWGGSLLGPWLSSGLKPLK

SEQ ID NO: 164 - protein, 18 aa, Artificial Sequence
  Description of Artificial Sequence Synthetic peptide
ESADLPPKGFQASYGKDE

SEQ ID NO: 165 - protein, 20 aa, Artificial Sequence
  Description of Artificial Sequence Synthetic peptide
TYPEADSFPSWAAAHRKGSS

SEQ ID NO: 166 - protein, 24 aa, Artificial Sequence
  Description of Artificial Sequence Synthetic peptide
RDAFESLFQSFETWHRGDALSRLD

SEQ ID NO: 167 - protein, 16 aa, Artificial Sequence
  Description of Artificial Sequence Synthetic peptide
APPTRLAPPQRPPPPS

SEQ ID NO: 168 - protein, 16 aa, Artificial Sequence
  Description of Artificial Sequence Synthetic peptide
MWSSINCIICACVKGR

SEQ ID NO: 169 - protein, 20 aa, Artificial Sequence
  Description of Artificial Sequence Synthetic peptide
RRWRKLYRANSHLFQAKRFN

SEQ ID NO: 170 - protein, 17 aa, Artificial Sequence
  Description of Artificial Sequence Synthetic peptide
AVVSYVVADMKEMLPRS

SEQ ID NO: 171 - protein, 23 aa, Artificial Sequence
  Description of Artificial Sequence Synthetic peptide
LNGSEPTGAYSIKGFFADYEIPN

SEQ ID NO: 172 - protein, 16 aa, Artificial Sequence
  Description of Artificial Sequence Synthetic peptide
GQLFPYRARIEIKNKF

SEQ ID NO: 173 - protein, 16 aa, Artificial Sequence
  Description of Artificial Sequence Synthetic peptide
QFDQLYRTKPGMTMSC

SEQ ID NO: 174 - protein, 15 aa, Artificial Sequence
  Description of Artificial Sequence Synthetic peptide
PRTESSDVADQLWAQ

SEQ ID NO: 175 - protein, 22 aa, Artificial Sequence
  Description of Artificial Sequence Synthetic peptide
RDILELGGPEEDAASGTISKKD

SEQ ID NO: 176 - protein, 8 aa, Artificial Sequence
  Description of Artificial Sequence Synthetic peptide
SQAGTVTL

SEQ ID NO: 177 - protein, 8 aa, Artificial Sequence
  Description of Artificial Sequence Synthetic peptide
QSRSSPTL

SEQ ID NO: 178 - protein, 8 aa, Artificial Sequence
  Description of Artificial Sequence Synthetic peptide
KVTNCFSV

SEQ ID NO: 179 - protein, 8 aa, Artificial Sequence
  Description of Artificial Sequence Synthetic peptide
TWHRGDAL

SEQ ID NO: 180 - protein, 9 aa, Artificial Sequence
  Description of Artificial Sequence Synthetic peptide
SSQAGTVTL

SEQ ID NO: 181 - protein, 9 aa, Artificial Sequence
  Description of Artificial Sequence Synthetic peptide
AQSRSSPTL

SEQ ID NO: 182 - protein, 9 aa, Artificial Sequence
  Description of Artificial Sequence Synthetic peptide
RRACGARTL

SEQ ID NO: 183 - protein, 10 aa, Artificial Sequence
  Description of Artificial Sequence Synthetic peptide
ASSQAGTVTL

SEQ ID NO: 184 - protein, 10 aa, Artificial Sequence
  Description of Artificial Sequence Synthetic peptide
RAPEIILGLL

SEQ ID NO: 185 - protein, 10 aa, Artificial Sequence
  Description of Artificial Sequence Synthetic peptide
EAQSRSSPTL

SEQ ID NO: 186 - protein, 10 aa, Artificial Sequence
  Description of Artificial Sequence Synthetic peptide
NTSALPLVSL

SEQ ID NO: 187 - protein, 10 aa, Artificial Sequence
  Description of Artificial Sequence Synthetic peptide
DSSIPSAATL

SEQ ID NO: 188 - protein, 11 aa, Artificial Sequence
  Description of Artificial Sequence Synthetic peptide
SAGENPLEEEE

SEQ ID NO: 189 - protein, 11 aa, Artificial Sequence
  Description of Artificial Sequence Synthetic peptide
NSPSTPTEQRI

SEQ ID NO: 190 - protein, 11 aa, Artificial Sequence
  Description of Artificial Sequence Synthetic peptide
FEAPGFNTTTA

SEQ ID NO: 191 - protein, 13 aa, Artificial Sequence
  Description of Artificial Sequence Synthetic peptide
AKFVAAWTLKAAA

SEQ ID NO: 192 - protein, 13 aa, Artificial Sequence
  Description of Artificial Sequence Synthetic peptide
AKFVAAWTLKAAA

SEQ ID NO: 193 - protein, 13 aa, Artificial Sequence
  Description of Artificial Sequence Synthetic peptide
AKFVAAWTLKAAA

SEQ ID NO: 194 - protein, 25 aa, Artificial Sequence
  Description of Artificial Sequence Synthetic peptide
LFMLTFSTSPGLESPVESFIAFLLI

SEQ ID NO: 195 - protein, 25 aa, Artificial Sequence
  Description of Artificial Sequence Synthetic peptide
PVVNGESNSLTSSVLYPTASLVSQN

SEQ ID NO: 196 - protein, 20 aa, Artificial Sequence
  Description of Artificial Sequence Synthetic peptide
SARKCLAKQAEDAAREKSCK

SEQ ID NO: 197 - protein, 23 aa, Artificial Sequence
  Description of Artificial Sequence Synthetic peptide
TNSAIQVLESSSLSLTDSLNGNS

SEQ ID NO: 198 - protein, 21 aa, Artificial Sequence
  Description of Artificial Sequence Synthetic peptide
PTLPQPASHFSPPPPPPPLPP

SEQ ID NO: 199 - protein, 20 aa, Artificial Sequence
  Description of Artificial Sequence Synthetic peptide
FINPIFEFSQAMRRLGLDDA

SEQ ID NO: 200 - protein, 26 aa, Artificial Sequence
  Description of Artificial Sequence Synthetic peptide
STVQKRELVNSASMKQALIASARRLP

SEQ ID NO: 201 - protein, 20 aa, Artificial Sequence
  Description of Artificial Sequence Synthetic peptide
EDTGQDMLALFLRTNRQAAK

SEQ ID NO: 202 - protein, 18 aa, Artificial Sequence
  Description of Artificial Sequence Synthetic peptide
YGRTVVPFLVPGTSQLGQ

SEQ ID NO: 203 - protein, 22 aa, Artificial Sequence
  Description of Artificial Sequence Synthetic peptide
YIHTSVSQDFSQSVPGTTSSPL

SEQ ID NO: 204 - protein, 23 aa, Artificial Sequence
  Description of Artificial Sequence Synthetic peptide
VVVSSLLLQEEELLAGGKPGADG

SEQ ID NO: 205 - protein, 21 aa, Artificial Sequence
  Description of Artificial Sequence Synthetic peptide
LSNQQPGLMVSFSLRLFPLFV

SEQ ID NO: 206 - protein, 21 aa, Artificial Sequence
  Description of Artificial Sequence Synthetic peptide
PVDNSSAKIVLKISYASKVKE

SEQ ID NO: 207 - protein, 20 aa, Artificial Sequence
  Description of Artificial Sequence Synthetic peptide
QIMLRSGVDLSVTDKREWRP

SEQ ID NO: 208 - protein, 26 aa, Artificial Sequence
  Description of Artificial Sequence Synthetic peptide
RNQHQRLLKNMGAHLVVLDLLQIPYE

SEQ ID NO: 209 - protein, 30 aa, Artificial Sequence
  Description of Artificial Sequence Synthetic peptide
QPASAAKCTGDFSPSLEKLVKSGNPLQPVS

SEQ ID NO: 210 - protein, 25 aa, Artificial Sequence
  Description of Artificial Sequence Synthetic peptide
TPSAPEGYDLKIGLFLAPRRGSLPD

SEQ ID NO: 211 - protein, 21 aa, Artificial Sequence
  Description of Artificial Sequence Synthetic peptide
MILASPRYVDQVTEFLQQKLK

SEQ ID NO: 212 - protein, 21 aa, Artificial Sequence
  Description of Artificial Sequence Synthetic peptide
VGRAWDAITDHLVGLCISKSP

SEQ ID NO: 213 - protein, 21 aa, Artificial Sequence
  Description of Artificial Sequence Synthetic peptide
FLSPGQLLQEPRTSLLIINNT

SEQ ID NO: 214 - protein, 23 aa, Artificial Sequence
  Description of Artificial Sequence Synthetic peptide
RSTTKSPGPSRHSKSPASTSSVN

SEQ ID NO: 215 - protein, 20 aa, Artificial Sequence
  Description of Artificial Sequence Synthetic peptide
KLESTVGSPKKPLSDLGKLS

SEQ ID NO: 216 - protein, 25 aa, Artificial Sequence
  Description of Artificial Sequence Synthetic peptide
GRPRMMGTGLSPYPEHLTSPLSPAQ

SEQ ID NO: 217 - protein, 20 aa, Artificial Sequence
  Description of Artificial Sequence Synthetic peptide
PLNPPASTAFSQEPHSGSPA

SEQ ID NO: 218 - protein, 27 aa, Artificial Sequence
  Description of Artificial Sequence Synthetic peptide
AAFVTPDQKYSMDNTLHTPTPFKNALE

SEQ ID NO: 219 - protein, 21 aa, Artificial Sequence
  Description of Artificial Sequence Synthetic peptide
RNLYISGFSLCFWLVLRRLVT

SEQ ID NO: 220 - protein, 19 aa, Artificial Sequence
  Description of Artificial Sequence Synthetic peptide
NAQLVVMILDGQNMAEDEF

SEQ ID NO: 221 - protein, 18 aa, Artificial Sequence
  Description of Artificial Sequence Synthetic peptide
RVLCVGTLRLHELIKLTA

SEQ ID NO: 222 - protein, 22 aa, Artificial Sequence
  Description of Artificial Sequence Synthetic peptide
HKDAVTCVNFSSSGHLLASGSR

SEQ ID NO: 223 - protein, 23 aa, Artificial Sequence
  Description of Artificial Sequence Synthetic peptide
LGNLAQFWECCLSSSGDADGESF

SEQ ID NO: 224 - protein, 18 aa, Artificial Sequence
  Description of Artificial Sequence Synthetic peptide
HIAGTSGFSLSFHSTVIN

SEQ ID NO: 225 - protein, 26 aa, Artificial Sequence
  Description of Artificial Sequence Synthetic peptide
LPTIKYLTLRLQDYLSLSHLVVYVPS

SEQ ID NO: 226 - protein, 28 aa, Artificial Sequence
  Description of Artificial Sequence Synthetic peptide
RLVHSGSGCRSPFLGSDLTFATRTGSRQ

SEQ ID NO: 227 - protein, 22 aa, Artificial Sequence
  Description of Artificial Sequence Synthetic peptide
PPKSPGPHSEKEDEAEPSTVPG

SEQ ID NO: 228 - protein, 20 aa, Artificial Sequence
  Description of Artificial Sequence Synthetic peptide
PFLHTVSKTRLFEYLRLTSL

SEQ ID NO: 229 - protein, 8 aa, Artificial Sequence
  Description of Artificial Sequence Synthetic peptide
TGQDMLAL

SEQ ID NO: 230 - protein, 8 aa, Artificial Sequence
  Description of Artificial Sequence Synthetic peptide
YKTDLHSL

SEQ ID NO: 231 - protein, 9 aa, Artificial Sequence
  Description of Artificial Sequence Synthetic peptide
LAQFWECCL

SEQ ID NO: 232 - protein, 9 aa, Artificial Sequence
  Description of Artificial Sequence Synthetic peptide
SLLLQEEEL

SEQ ID NO: 233 - protein, 9 aa, Artificial Sequence
  Description of Artificial Sequence Synthetic peptide
CTGDFSPSL

SEQ ID NO: 234 - protein, 8 aa, Artificial Sequence
  Description of Artificial Sequence Synthetic peptide
AQFWECCL

SEQ ID NO: 235 - protein, 8 aa, Artificial Sequence
  Description of Artificial Sequence Synthetic peptide
LLLQEEEL

SEQ ID NO: 236 - protein, 8 aa, Artificial Sequence
  Description of Artificial Sequence Synthetic peptide
TGDFSPSL

SEQ ID NO: 237 - protein, 8 aa, Artificial Sequence
  Description of Artificial Sequence Synthetic peptide
QVLESSSL

SEQ ID NO: 238 - protein, 8 aa, Artificial Sequence
  Description of Artificial Sequence Synthetic peptide
NSLTSSVL

SEQ ID NO: 239 - protein, 8 aa, Artificial Sequence
  Description of Artificial Sequence Synthetic peptide
NSSAKIVL

SEQ ID NO: 240 - protein, 9 aa, Artificial Sequence
  Description of Artificial Sequence Synthetic peptide
NSASMKQAL

SEQ ID NO: 241 - protein, 10 aa, Artificial Sequence
  Description of Artificial Sequence Synthetic peptide
AIQVLESSSL

SEQ ID NO: 242 - protein, 9 aa, Artificial Sequence
  Description of Artificial Sequence Synthetic peptide
LGIGGLQDL

SEQ ID NO: 243 - protein, 8 aa, Artificial Sequence
  Description of Artificial Sequence Synthetic peptide
LSPYPEHL

SEQ ID NO: 244 - protein, 13 aa, Artificial Sequence
  Description of Artificial Sequence Synthetic peptide
AKFVAAWTLKAAA

SEQ ID NO: 245 - protein, 13 aa, Artificial Sequence
  Description of Artificial Sequence Synthetic peptide
AKFVAAWTLKAAA

SEQ ID NO: 246 - protein, 20 aa, Artificial Sequence
  Description of Artificial Sequence Synthetic peptide
SPRVAPGSAPPWPALRSLLH

SEQ ID NO: 247 - protein, 19 aa, Artificial Sequence
  Description of Artificial Sequence Synthetic peptide
VLGTSAPGSSRLAAVDLGG

SEQ ID NO: 248 - protein, 19 aa, Artificial Sequence
  Description of Artificial Sequence Synthetic peptide
ARPPGGSGPLRVLIPDLQL

SEQ ID NO: 249 - protein, 17 aa, Artificial Sequence
  Description of Artificial Sequence Synthetic peptide
PSAPQQEGVASKEKEEV

SEQ ID NO: 250 - protein, 22 aa, Artificial Sequence
  Description of Artificial Sequence Synthetic peptide
TSQAYNALTLVVTSCKNFKVRI

SEQ ID NO: 251 - protein, 22 aa, Artificial Sequence
  Description of Artificial Sequence Synthetic peptide
GENSVSSSPSASSTAALNTAAA

SEQ ID NO: 252 - protein, 21 aa, Artificial Sequence
  Description of Artificial Sequence Synthetic peptide
FETTTGFDPHSGTPLSDHEAL

SEQ ID NO: 253 - protein, 20 aa, Artificial Sequence
  Description of Artificial Sequence Synthetic peptide
FQSLCQAPPLLKDKVLTALE

SEQ ID NO: 254 - protein, 20 aa, Artificial Sequence
  Description of Artificial Sequence Synthetic peptide
VVPGNVTLSVVGSTSVPLSS

SEQ ID NO: 255 - protein, 16 aa, Artificial Sequence
  Description of Artificial Sequence Synthetic peptide
RPGEDPSLHGIVKEQL

SEQ ID NO: 256 - protein, 21 aa, Artificial Sequence
  Description of Artificial Sequence Synthetic peptide
SPAESCDLLGAIQTCIRKSLG

SEQ ID NO: 257 - protein, 25 aa, Artificial Sequence
  Description of Artificial Sequence Synthetic peptide
MKMASFLAFLLLNFHVCLLLLQLLM

SEQ ID NO: 258 - protein, 21 aa, Artificial Sequence
  Description of Artificial Sequence Synthetic peptide
SENQQPGAPNTPTHPAPPGLH

SEQ ID NO: 259 - protein, 29 aa, Artificial Sequence
  Description of Artificial Sequence Synthetic peptide
HLINYQDDAELATHALPELTKLLNDEDPV

SEQ ID NO: 260 - protein, 17 aa, Artificial Sequence
  Description of Artificial Sequence Synthetic peptide
LIVENVHFQAHKALLAA

SEQ ID NO: 261 - protein, 18 aa, Artificial Sequence
  Description of Artificial Sequence Synthetic peptide
STAPAEATLPKPGEAEAP

SEQ ID NO: 262 - protein, 26 aa, Artificial Sequence
  Description of Artificial Sequence Synthetic peptide
PYSGLGGVGDPYAPLMVLMCRVCLED

SEQ ID NO: 263 - protein, 16 aa, Artificial Sequence
  Description of Artificial Sequence Synthetic peptide
DFYLRGAVALSVRPIS

SEQ ID NO: 264 - protein, 15 aa, Artificial Sequence
  Description of Artificial Sequence Synthetic peptide
TDVDPQSAVMQEEIF

SEQ ID NO: 265 - protein, 21 aa, Artificial Sequence
  Description of Artificial Sequence Synthetic peptide
NSLQNQALQTLQERLHEADAT

SEQ ID NO: 266 - protein, 21 aa, Artificial Sequence
  Description of Artificial Sequence Synthetic peptide
GAEDSIDSPSACPLSTGCPAL

SEQ ID NO: 267 - protein, 22 aa, Artificial Sequence
  Description of Artificial Sequence Synthetic peptide
RFIGPLPREGSVGSTSDYVSQS

SEQ ID NO: 268 - protein, 22 aa, Artificial Sequence
  Description of Artificial Sequence Synthetic peptide
IQSIYGGLPKVPAKPKEPTIPH

SEQ ID NO: 269 - protein, 26 aa, Artificial Sequence
  Description of Artificial Sequence Synthetic peptide
FWGILGFPALYTHLPAFLEWTLCLLS

SEQ ID NO: 270 - protein, 20 aa, Artificial Sequence
  Description of Artificial Sequence Synthetic peptide
VDLKFPASVPTGAQDLISKL

SEQ ID NO: 271 - protein, 16 aa, Artificial Sequence
  Description of Artificial Sequence Synthetic peptide
LAPGQPFLSSQGSLCI

SEQ ID NO: 272 - protein, 18 aa, Artificial Sequence
  Description of Artificial Sequence Synthetic peptide
RVGDLSPKQKEALAKPEA

SEQ ID NO: 273 - protein, 19 aa, Artificial Sequence
  Description of Artificial Sequence Synthetic peptide
LAVRWFFAHSSDSQEALMV

SEQ ID NO: 274 - protein, 20 aa, Artificial Sequence
  Description of Artificial Sequence Synthetic peptide
YSGIQESSSASPLSIKKCPI

SEQ ID NO: 275 - protein, 16 aa, Artificial Sequence
  Description of Artificial Sequence Synthetic peptide
LIKPPAHTSAILTVLR

SEQ ID NO: 276 - protein, 17 aa, Artificial Sequence
  Description of Artificial Sequence Synthetic peptide
MSYELKCAQELSQKQDG

SEQ ID NO: 277 - protein, 17 aa, Artificial Sequence
  Description of Artificial Sequence Synthetic peptide
QVHQCSVLLVATGLSVP

SEQ ID NO: 278 - protein, 22 aa, Artificial Sequence
  Description of Artificial Sequence Synthetic peptide
RSLTLEPDPIVVPGNVTLSVVG

SEQ ID NO: 279 - protein, 19 aa, Artificial Sequence
  Description of Artificial Sequence Synthetic peptide
LDRQHVQHQLLVILKELRK

SEQ ID NO: 280 - protein, 25 aa, Artificial Sequence
  Description of Artificial Sequence Synthetic peptide
TVDMLQCLRFPGLALPHTRAPSPLG

SEQ ID NO: 281 - protein, 11 aa, Artificial Sequence
  Description of Artificial Sequence Synthetic peptide
SAPGSSRLAAV

SEQ ID NO: 282 - protein, 8 aa, Artificial Sequence
  Description of Artificial Sequence Synthetic peptide
VALSVRPI

SEQ ID NO: 283 - protein, 8 aa, Artificial Sequence
  Description of Artificial Sequence Synthetic peptide
SAVMQEEI

SEQ ID NO: 284 - protein, 9 aa, Artificial Sequence
  Description of Artificial Sequence Synthetic peptide
AAIQEKKEI

SEQ ID NO: 285 - protein, 11 aa, Artificial Sequence
  Description of Artificial Sequence Synthetic peptide
VSPDIFMQSHL

SEQ ID NO: 286 - protein, 8 aa, Artificial Sequence
  Description of Artificial Sequence Synthetic peptide
QAYNALTL

SEQ ID NO: 287 - protein, 9 aa, Artificial Sequence
  Description of Artificial Sequence Synthetic peptide
RVLIPDLQL

SEQ ID NO: 288 - protein, 8 aa, Artificial Sequence
  Description of Artificial Sequence Synthetic peptide
APGSSRLA

SEQ ID NO: 289 - protein, 9 aa, Artificial Sequence
  Description of Artificial Sequence Synthetic peptide
STAPAEATL

SEQ ID NO: 290 - protein, 11 aa, Artificial Sequence
  Description of Artificial Sequence Synthetic peptide
GALPVASPASL

SEQ ID NO: 291 - protein, 10 aa, Artificial Sequence
  Description of Artificial Sequence Synthetic peptide
SAPGSSRLAA

SEQ ID NO: 292 - protein, 11 aa, Artificial Sequence
  Description of Artificial Sequence Synthetic peptide
TSAPGSSRLAA

SEQ ID NO: 293 - protein, 8 aa, Artificial Sequence
  Description of Artificial Sequence Synthetic peptide
SLCQAPPL

SEQ ID NO: 294 - protein, 11 aa, Artificial Sequence
  Description of Artificial Sequence Synthetic peptide
MSYELKCAQEL

SEQ ID NO: 295 - protein, 8 aa, Artificial Sequence
  Description of Artificial Sequence Synthetic peptide
LAPGQPFL

SEQ ID NO: 296 - protein, 13 aa, Artificial Sequence
  Description of Artificial Sequence Synthetic peptide
AKFVAAWTLKAAA

SEQ ID NO: 297 - protein, 13 aa, Artificial Sequence
  Description of Artificial Sequence Synthetic peptide
AKFVAAWTLKAAA